(19) United States
(12) Patent Application Publication
(10) Pub. No.: US 2022/0118215 A1
(43) Pub. Date: Apr. 21, 2022

(54) CLOSED-CIRCUIT MIXED GAS DELIVERY SYSTEMS AND METHODS

(71) Applicant: SaiOx, Inc., Tucson, AZ (US)

(72) Inventors: MANUEL TERAN, Tucson, AZ (US); Erik HERRMANN, Mesa, AZ (US); Candice EATON, Tucson, AZ (US)

(21) Appl. No.: 17/073,021

(22) Filed: Oct. 16, 2020

(51) Int. Cl. A61M 16/22; B01D 53/14; B01D 53/04; B01D 53/22; B01D 53/62; B01F 15/04; A61M 16/20; A61M 16/00; A61M 16/08; A61M 16/10

(52) U.S. Cl. CPC A61M 16/22; A61M 2202/0225; B01D 53/04; B01D 53/22; B01D 53/1493; B01D 53/62; B01F 15/0412; A61M 16/209; A61M 16/022; A61M 16/0833; A61M 16/1045; A61M 16/105; A61M 16/0078; B01D 2253/102; B01D 2253/108; B01D 2252/204; B01D 2257/504; B01D 2251/304; B01D 2251/306; B01D 2251/302; B01D 2251/404; B01D 2251/604; B01F 2215/0034; A61M 2205/3379; A61M 2205/3334; A61M 2202/0208; A61M 2202/025; B01D 53/1475

(57) ABSTRACT

The present disclosure is directed to systems and methods of providing a mixed-gas inhalant to a patient via a gas recirculation loop. The gas recirculation loop receives a first mixed-gas exhalant having a first carbon dioxide concentration from the patient, one or more carbon dioxide removal devices discharge a second mixed-gas exhalant having a second carbon dioxide concentration that is less than the first carbon dioxide concentration. The second mixed-gas exhalant is combined with a mixed-gas supply to provide a mixed-gas inhalant. The mied-gas supply includes a first gas and a second gas. The mixed-gas supply is pressure and flow controlled to produce a mixed-gas inhalant having a defined composition delivered to the patient at a defined volumetric flow rate. The first gas may include a gas containing oxygen and the second gas may include a gas mixture containing a noble or inert gas and oxygen.

### TECHNICAL FIELD

The present disclosure relates to systems and methods for alleviating the symptoms of pulmonary diseases such as chronic obstructive pulmonary disease (COPD).

### BACKGROUND

Chronic obstructive pulmonary disease (COPD) is a chronic, slowly progressive disorder characterized by airflow obstruction (associated with aberrant airway inflammation) and airway remodeling. COPD symptoms can present with airway tissue damage. Airflow limitation is slowly progressive, leading to dyspnoea and limitations of physical exercise capacities. However, impairment is not restricted to the lungs, as COPD patients are also at higher risk for systemic failures including cardiovascular diseases.

The clinical presentation of COPD can vary in severity from simple chronic bronchitis without disability to a severely disabled state with chronic respiratory failure. The diagnosis of COPD is usually suggested by symptoms, but is typically only established by quantitative measurements, preferably using spirometry.

COPD is the fourth leading causes of death worldwide and the only major disease with an increasing death rate. By 2020, it is estimated that only ischemic heart disease and cerebrovascular disease will account for a higher mortality among the world's population. Prevalence and hospitalization rates have increased dramatically over the past years. According to statistics from the National Institute of Health (NIH), 12 million adults in the U.S. are diagnosed with COPD, and 120,000 die from it each year. An additional 12 million adults in the U.S. are thought to have undiagnosed COPD. COPD accounts for 1.5 million emergency room visits and over 700,000 hospitalizations annually, with an estimated cost to the US healthcare system of over $32.1 billion in 2001. COPD death rates for women have risen steadily. Today, more women than men die from COPD each year.

There is a scarcity of available information and extremely low public awareness about COPD. There are few efficacious alternative treatments available. Lack of awareness and the insidious nature of the disease have been major contributing factors to the low diagnostic rates seen in COPD. As the symptoms of the disease occur with the onset of middle age, many patients dismiss symptoms such as breathlessness upon exertion as old age. Smokers, who constitute the majority of COPD sufferers, also commonly dismiss symptoms, such as chronic cough, as something to be expected because of smoking, rather than an indication of a serious underlying problem.

The Nocturnal Oxygen Therapy Trial supported by the NIH showed that patients with advanced COPD live longer with long-term oxygen therapy. Clinical trials sponsored by the National Heart, Lung, and Blood Institute and the Centers for Medicare and Medicaid Services are investigating the effectiveness of oxygen treatment for increasing life expectancy in patients with moderate COPD.

Heliox is a breathing gas composed of a mixture of Helium (He) and Oxygen (O2). Heliox generates less airway resistance than air and requires less mechanical energy to ventilate the lungs, reducing the Work of Breathing (WOB). While oxygen alone may reduce breathlessness, heliox reduces resistance in the lungs during exhalation, which allows COPD patients to exhale more air. This means the lungs can better eliminate carbon dioxide from the body. According to results of a randomized, crossover trial, a combination of helium and oxygen improves walking distance in patients with COPD. Wedzicha, American Journal of Respiratory and Critical Care Medicine, 173(8):825-826 (2006). In another, smaller study, giving heliox during exercise allowed a greater amount of air to be inhaled by the lungs and reduced shortness of breath scores compared to those who were on room air. Heliox induced a state of hyperventilation, which reduced carbon dioxide levels in the blood of patients studied, increasing exercise capacity. See Palange et al., J Appl Physiol, 97:1637-1642 (2003).

Although medical uses of heliox have been known since the 1930s, it is typically only administered in settings such as a hospital or modern specialist respiratory centers. Systems used in these settings rely on an invasive ventilator system or non-invasive open-circuit breathing systems to deliver heliox. However, open-circuit breathing systems are inefficient for delivery of heliox because a portion of the gas escapes into the atmosphere prior to reaching a patient's lungs and helium is lost to the atmosphere upon exhalation. This results in as many as four to six full-sized tanks of heliox being required for 24 hours of treatment. Helium is over 13 times as expensive as oxygen. Therefore, current heliox treatments are inconvenient and cost-prohibitive for many patients.

Closed-circuit breathing apparatuses have been developed for applications requiring complete respiratory protection in potentially hazardous conditions, such as decontamination, urban search and rescue, or biohazard situations. Exhaled carbon dioxide is typically removed by an in-line carbon dioxide scrubbing device. These systems typically deliver only pure oxygen or standard or enhanced breathing air mixture, similar to that used during scuba diving. Currently available closed-circuit breathing apparatuses are not configured to regulate a mixture of heliox. Further, current closed-circuit breathing apparatuses can only function for limited durations (e.g. 4 hours or less). Thus, current closed-circuit breathing apparatuses are not suitable for long term (i.e. overnight) use.

The amount of oxygen within the closed-circuit is regulated by standard in-line and/or tank regulator that is manually adjusted prior to or during use. Therefore, a user must be conscious in order to operate the breathing apparatus. Alternatively, an attendant trained in the use of the mixed-gas delivery system is typically present for sleeping or unconscious individuals.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1A is a schematic diagram of an illustrative mixed-gas delivery system that includes a self-contained, pneumatically actuated, mixed-gas recirculation system to reduce the concentration of carbon dioxide in an exhalant received from a patient and recycle at least a portion of the exhalant as an inhalant to the patient, a supply gas system that includes at least a first gas reservoir and a second gas reservoir, and a breathing circuit to provide the inhalant to the patient, in accordance with at least one embodiment described herein;

FIG. 1B is a schematic diagram of an illustrative mixed-gas delivery system similar to the mixed-gas delivery system depicted in FIG. 1A but in which control circuitry, such as processor circuitry, is used to monitor and/or control the operation of the mixed-gas recirculation system, in accordance with at least one embodiment described herein;

FIG. 2A is a schematic diagram of a replaceable first module that includes the gas recirculation loop in which the components depicted are replaced for each new patient, in accordance with at least one embodiment described herein;

FIG. 2B is an external elevation view of the replaceable first module depicted in FIG. 2A, in accordance with at least one embodiment described herein;

FIG. 2C is a detail view of the illustrative transparent panel that allows visual confirmation of inflation of the one or more volumetric flow devices, in accordance with at least one embodiment described herein;

FIG. 3A is a schematic diagram of a system in which the single-use or replaceable first module depicted in FIGS. 2A-2C operably couples to a multi-use second module that includes the instruments, the gas blending and flow control devices, and the one or more pressure reducing devices to manually and/or pneumatically control the operation of the gas recirculation loop, in accordance with at least one embodiment described herein;

FIG. 3B is a schematic diagram of another system in which the second module includes local control circuitry and a local user interface to electronically control the operation of at least a portion of the gas recirculation loop, in accordance with at least one embodiment described herein;

FIG. 3C is a schematic diagram of yet another system in which the second module includes network connected remote control circuitry and a remote user interface to electronically control the operation of at least a portion of the gas recirculation loop, in accordance with at least one embodiment described herein;

FIG. 4 is a schematic diagram of an illustrative mixed-gas delivery system similar to that depicted in FIGS. 1A and 1n which a purge valve has been installed in the gas recirculation loop, in accordance with at least one embodiment described herein;

FIG. 5 is a high-level flow diagram of an illustrative mixed-gas recirculation method, in accordance with at least one embodiment described herein. The method 400 employs a

Although the following Detailed Description will proceed with reference being made to illustrative embodiments, many alternatives, modifications and variations thereof will be apparent to those skilled in the art.

### DETAILED DESCRIPTION

The mixed-gas delivery systems disclosed herein offer the advantage of portability and do not require an external power source, beneficially making possible the use of such mixed-gas delivery systems in remote areas where an external power supply may be unreliable or unavailable. The mixed-gas delivery systems disclosed herein may be constructed as a single unitary system with individually replaceable components. The mixed-gas delivery systems disclosed herein include a closed-loop system that receives exhalant from a patient, removes at least a portion of the carbon dioxide from the exhalant, mixes the exhalant with a mixed-gas supply to produce an inhalant and volumetrically feeds the inhalant to the patient. The mixed-gas delivery systems disclosed herein also include biological HEPA filters to protect multi-use components from exposure to the patient, overpressure and vacuum relief devices, backflow prevention devices, instrumentation, mixed-gas supply regulators, and one or more mixed-gas flow control devices. Beneficially, the mixed-gas delivery systems disclosed herein may use any type of gas supply including cylinders for use in residential or commercial environments and/or bulk delivery systems typically found in institutional environments. Additionally, the systems and methods disclosed herein do not routinely produce an emission to the surrounding environment, thereby limiting or even preventing the spread of biological contaminants from the patient to the surrounding environment, other patients, or healthcare personnel.

Additionally or alternatively, the mixed-gas delivery systems disclosed herein may be modularly constructed, for example as a single use first module that includes several relatively low-cost mixed-gas recirculation system components and a multi-use second module that includes several additional, relatively high-cost, non-patient contact, components. Beneficially, the first module may include some or all of patient-contact components and is easily replaceable. The second module may include some or all of the non-patient contact components. The first module operably couples and detachably attaches to the second module. Beneficially, such modular construction may reduce the down time between patients and may reduce the likelihood of error in setting up the mixed-gas delivery system, improving both reliability and availability.

The single-use first module includes one or more ports for supplying a mixed-gas inhalant to a patient and receiving the exhalent from the patient, one or more instrumentation ports for coupling measurement devices to the closed-loop, mixed-gas system, and one or more ports to supply one or more gases from respective external sources to the recirculated, closed-loop, mixed-gas system. Other single-use components in the first module include one or more biological HEPA, a visual inhalant volumetric flow device, a carbon dioxide scrubber to reduce the concentration of carbon dioxide in the patient's exhalant to an acceptable level, one or more overpressure relief devices, one or more vacuum relief devices, and one or more backflow prevention devices. The multi-use second module includes instrumentation to monitor one or more physical (e.g., pressure, temperature, volume) parameters and/or one or more chemical (e.g., mixed-gas ratio, gas concentration) parameters of the inhalant supplied to the patient via the mixed-gas recirculation system. The second module also includes one or more regulators to control the pressure and/or flowrate of the supply gases provided to the mixed-gas recirculation system.

A mixed-gas recirculation system is provided. The mixed-gas recirculation system may include: a gas recirculation loop that includes: a first connection to receive a first mixed-gas exhalant from a patient, the first mixed-gas exhalant having carbon dioxide at a first concentration; a carbon dioxide removal device to remove at least a portion of the carbon dioxide present in the first exhalant received from the patient to provide a second mixed-gas exhalant having carbon dioxide at a second volumetric concentration less than the first volumetric concentration; a mixer to combine the second exhalant with volumetrically controlled flowrate of a mixed-gas supply that includes a first gas and a second gas to provide a mixed-gas inhalant having a volumetric feed rate within a defined range; an overpressure protection device fluidly coupled to the gas recirculation loop; a vacuum protection device fluidly coupled to the gas recirculation loop; a volumetric flow device to measure a volume of the mixed-gas inhalant supplied to the patient; and a second connection to supply the mixed-gas inhalant to the patient. The system may also include: a first gas inlet to receive a regulated supply of the first gas having a first composition; a second gas inlet to receive a regulated supply of the second gas having a second composition; a first instrument fluidly coupled to the gas recirculation loop to measure one or more physical parameters of the mixed-gas inhalant; and a second instrument fluidly coupled to the gas recirculation loop to measure one or more compositional parameters of the mixed-gas inhalant.

A mixed-gas recirculation system is provided. The system may include: a housing having an external surface that includes a transparent panel disposed in at least a portion of the external surface, the housing including an internal void space; a gas recirculation loop disposed at least partially within the housing, the gas recirculation loop including: a first connection to receive a first mixed-gas exhalant from a patient, the first mixed-gas exhalant including carbon dioxide at a first concentration, the first connection disposed on the external surface of the housing; a carbon dioxide removal device to remove at least a portion of the carbon dioxide included in the first mixed-gas exhalant to provide a second mixed-gas exhalant including carbon dioxide at a second concentration less than the first concentration, the carbon dioxide removal subsystem disposed at least partially within the housing; at least one gas inlet to receive at least one of a first gas and a second gas, the at least one gas inlet disposed on the external surface of the housing; a mixer to mix the second mixed-gas exhalant with a mixed-gas supply that includes the first gas and the second gas to provide a mixed-gas inhalant having a volumetric flow rate to the patient within a defined range, the mixer disposed at least partially within the housing; an overpressure protection device fluidly coupled to the gas recirculation loop and disposed at least partially within the housing; a vacuum protection device fluidly coupled to the gas recirculation loop and disposed at least partially within the housing; and a second connection to supply the mixed-gas inhalant to a patient, the patient supply connection disposed on the external surface of the housing.

A method of supplying a mixed-gas to a patient is provided. The method may include: receiving, at a first connection to a gas recirculation loop, a first mixed-gas exhalant that includes carbon dioxide at a first concentration; removing, via a carbon dioxide removal device disposed in the gas recirculation loop, at least a portion of the carbon dioxide from the first mixed-gas exhalant to provide a second mixed-gas exhalant that includes carbon dioxide at a second concentration less than the first concentration; mixing the second mixed-gas exhalant with a mixed-gas supply that includes a first gas and a second gas to provide a mixed-gas inhalant at a volumetric flow rate within a defined range; measuring, via a volumetric flow device, the volumetric flow rate of the mixed-gas inhalant through the gas recirculation loop; measuring, via a first instrument, one or more physical parameters of the mixed-gas inhalant; measuring, via a second instrument, one or more compositional parameters of the mixed-gas inhalant; and supplying the inhalant to a second connection.

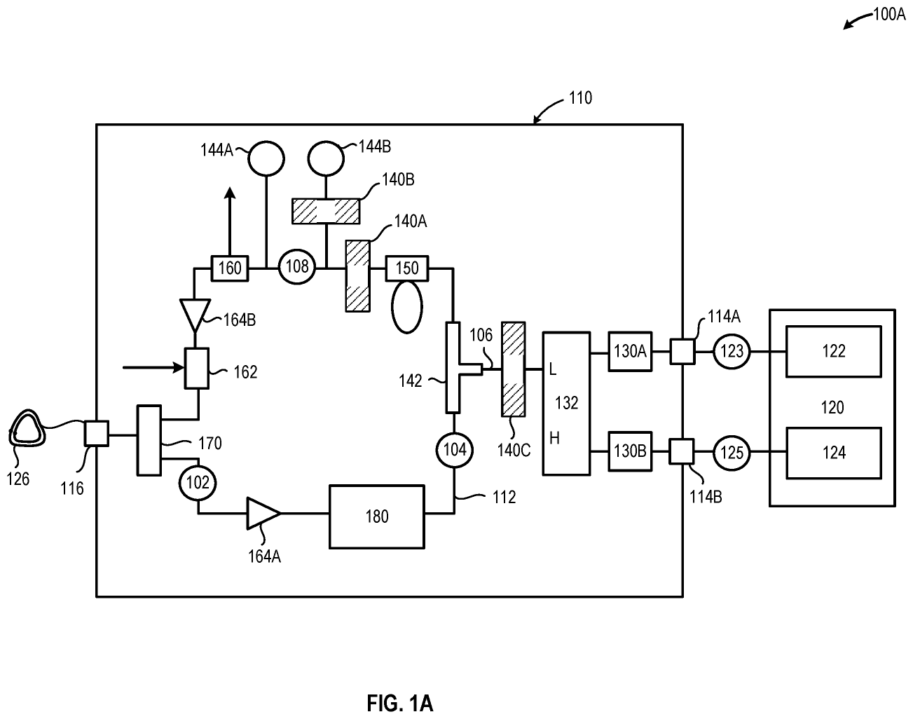

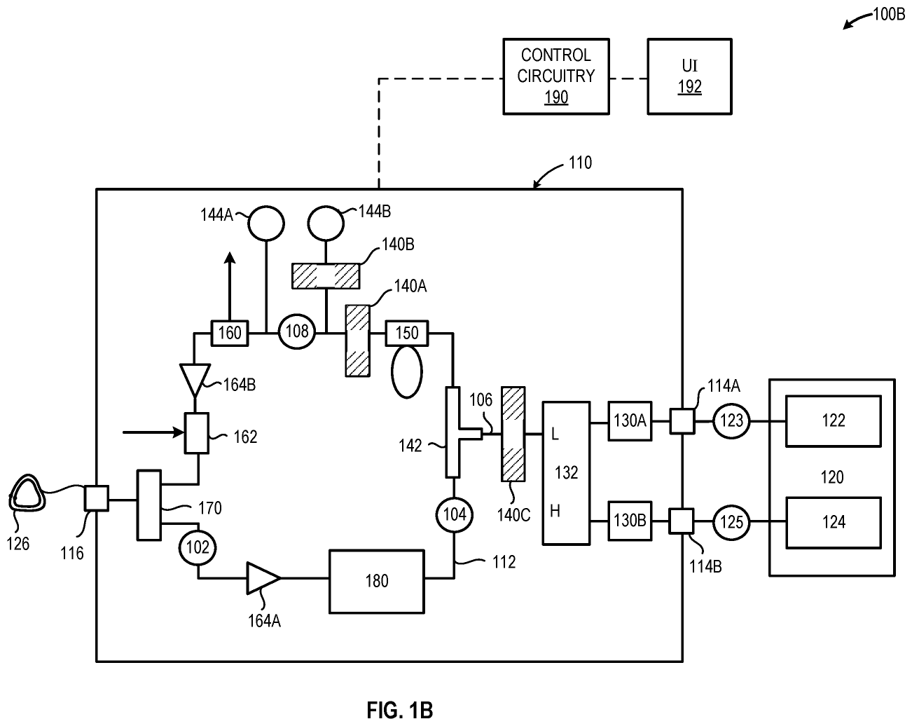

FIG. 1A is a schematic diagram of an illustrative mixed-gas delivery system 100A that includes a self-contained, pneumatically actuated, mixed-gas recirculation system 110 to reduce the concentration of carbon dioxide in an exhalant received from a patient and recycle at least a portion of the exhalant as an inhalant to the patient, a supply gas system 120 that includes at least a first gas reservoir 122 and a second gas reservoir 124, and a breathing circuit 126 to provide the inhalant to the patient, in accordance with at least one embodiment described herein. FIG. 1B is a schematic diagram of an illustrative mixed-gas delivery system 100B similar to the mixed-gas delivery system 100A but in which control circuitry 190, such as processor circuitry, is used to monitor and/or control the operation of the mixed-gas recirculation system 110, in accordance with at least one embodiment described herein.

Referring first to FIG. 1A, in embodiments the gas recirculation loop 112 may include a heat and moisture exchange device 170 to receive a first mixed-gas exhalant 102 from a patient. The first mixed-gas exhalant 102 flows through a first backflow prevention device 164A into a carbon dioxide removal device 180 where the concentration of carbon dioxide is decreased from a first, relatively higher concentration in the first mixed-gas exhalant 102, to a second, relatively lower concentration in a second mixed-gas exhalant 104. The second mixed-gas exhalant 104 flows from the carbon dioxide removal device 180 to a tee connection 142 where the second mixed-gas exhalant 104 is mixed with a flow and/or pressure controlled mixed-gas supply 106 to provide a mixed-gas inhalant 108. The mixed-gas inhalant 108 then passes through a volumetric flow device 150, a first biological HEPA filter 140A, an overpressure protection device 160, a second backflow prevention device 164B, and a vacuum protection device 162. At least a portion of the heat and moisture contained in the first, mixed-gas exhalant 102 from the patient is retained using a heat and moisture exchanger 170 and returned to the mixed-gas inhalant 108 to provide a warm, humidified mixed-gas inhalant 108 to the patient via the breathing circuit 126. The pressure of the mixed-gas supply 106 may be manually adjusted by the patient, an attendant, and/or medical professional using one or more pressure reducing devices 130A fluidly coupled to the first gas reservoir 122 and/or one or more pressure reducing devices 130B fluidly coupled to the second gas reservoir 124. The flow of mixed-gas supply 106 may be manually adjusted by the patient, an attendant, and/or medical professional using one or more gas blending and flow control devices 132 fluidly coupled to the one or more pressure reducing devices 130A and the one or more pressure reducing devices 130B.

The supply gas system 120 includes a first gas reservoir 122 and a second gas reservoir 124. The gas from each reservoir flows through pressure regulation devices 114A, 114B into a gas blending and flow control devices 132 and through a biological HEPA filter 140B prior to introduction to the tee connection 142. In some embodiments, the supply gas system 120 may include one or more gas reservoirs 122, 124, such as bulk and/or high pressure gas storage vessels, typically found in institutional settings, such as a hospital or emergency treatment setting. In other embodiments, the supply gas system 120 may include one or more gas reservoirs 122, 124, such as gas cylinders, typically found in residential or commercial settings. In at least one embodiment, the first gas reservoir 122 may contain oxygen or one or more oxygen containing gases and the second gas reservoir 124 may contain an oxygen/noble gas mixture, such as helium and oxygen (e.g., heliox).

In operation, a patient breathes through the breathing circuit 126. As the patient exhales, a mixed-gas first exhalant containing carbon dioxide at a first concentration is forced through one or more heat and moisture exchange (HME) devices 170, through one or more first backflow prevention devices 164A, and into one or more carbon dioxide removal devices 180. Within the one or more carbon dioxide removal devices 180 carbon dioxide is entrapped and a mixed-gas second exhalant 104 having a second carbon dioxide concentration lower than the first carbon dioxide concentration exits the one or more carbon dioxide removal devices 180. The mixed-gas second exhalant 104 is mixed with a fresh mixed-gas supply 106 using a mixing or combining device such as one or more tee connections 142 to provide a mixed-gas inhalant 108. The mixed-gas inhalant 108 flows into one or more volumetric flow devices 150, such as one or more breathing bags, which act as a counterlung to maintain air pressure within the gas recirculation loop 112.

As the patient inhales, the mixed-gas inhalant 108 is drawn from the one or more volumetric flow devices 150, through one or more first biological HEPA filters 140A and through one or more second backflow prevention devices 164B. After the air is drawn through the one or more second backflow prevention devices 164B, a first gas (e.g., oxygen) is drawn from the first gas reservoir 122 at a pressure and/or flow controlled rate and a second gas (e.g., a noble gas/oxygen mixture) is drawn from the second gas reservoir 124 at a pressure and/or flow controlled rate. One or more overpressure protection devices 160 relieve or release the mixed-gas inhalant 108 from the gas recirculation loop 112 in the event the pressure within the gas recirculation loop 112 exceeds a defined maximum threshold value. One or more vacuum protection devices 162 allow ambient air to enter the gas recirculation loop 112 in the event the pressure within the gas recirculation loop 112 falls below a defined minimum value. Beneficially, the entire system is controlled using the pressure within the gas recirculation loop 112 and does not require the use of an electrically powered controller or similar electrically powered and/or controlled devices. The above process is repeated for each breath drawn by the patient.

As the mixed-gas inhalant 108 moves through the gas recirculation loop 112, one or more analytical instruments 144A fluidly coupled to the gas recirculation loop 112 may provide an indication of one or more composition parameters (oxygen concentration, carbon dioxide concentration, noble gas concentration, etc.) in the mixed-gas inhalant. In addition, one or more gas loop physical parameter (e.g., pressure) instruments 144B may be fluidly coupled to the gas recirculation loop 112 through one or more second biological HEPA filters 140B. The information provided by the one or more instruments 144A, 144B may be used by the attendant or medical professional to adjust the pressure and/or flowrate of one or more of the first gas 123, the second gas 125, and/or the mixed-gas supply 106 into the gas recirculation loop 112.

In embodiments, the mixed-gas recirculation system 110 includes at least one patient mixed-gas supply connection 116 to operably and fluidly couple the breathing circuit 126 to the mixed-gas recirculation system 110. As used herein the term “breathing circuit” refers to a system of hoses or tubing that connect the patient to the mixed-gas recirculation system 110. The breathing circuit 126 carries a mixed-gas inhalant from the mixed-gas recirculation system 110 to the patient and returns the patient's carbon dioxide containing exhalant to the mixed-gas recirculation system 110 for carbon dioxide removal and replenishment with a mixed-gas from the supply gas system 120. The breathing circuit 126 contains an inspiratory portion for delivering the mixed-gas inhalant to the patient and an expiratory portion for delivering the carbon dioxide containing exhalant from the patient to the mixed-gas recirculation system 110. In at least some embodiments, the breathing circuit 126 may contain: (1) an inspiratory conduit that delivers a fresh mixed-gas inhalant from the mixed-gas recirculation system 110 to the patient; (2) an expiratory conduit that delivers carbon dioxide containing exhalant from the patient to the mixed-gas recirculation system 110; (3) a non-invasive mask or other suitable upper airway device to deliver the mixed-gas in and out of the lungs; and (4) a connector to connect the inspiratory and expiratory conduits to the upper airway device, such as a Y- or T-connector.

In embodiments, one or more additional components may be included in the breathing circuit 126. Such additional components are not depicted in FIG. 1A, and may include but are not limited to: a nebulizer to aerosolize and deliver additional therapeutic and/or diagnostic agents, and one or more humidification and/or dehumidification devices to control the moisture content of the mixed-gas inhalant delivered to the patient. In some embodiments, a humidification device may be used to deliver additional therapeutic and/or diagnostic agents, particularly those in particulate form that can be aerosolized.

In at least some embodiments, the inspiratory and expiratory conduits are made of disposable corrugated plastic or rubber hoses or tubing. Hoses generally have larger diameters than tubing. Hoses may have a corrugated design to allow bending of the hose while preventing twisting of the hose or cutting off of flow through the hose. Standard corrugated medical hoses may be used in the system and come with internal diameters of 6 mm, 10 mm, 15, mm, 19 mm, 22 mm, 25 mm, and 32 mm and in lengths ranging from 6 inches to 10 feet or more.

In embodiments, the breathing circuit 126 may include a volumetric flow indicator, such as a counterlung, chamber, or breathing bag. In such embodiments, the volumetric flow indicator may receive a volume of exhalant from the patient and provide a volume of inhalant to the inspiratory conduit. The volumetric flow indicator allows the patient to take in a deep breath without dropping the pressure in the mixed-gas recirculation system 110 to less than 0 centimeters of water (cm H₂0). In some embodiments, the breathing circuit 126 may contain one or more safety valves and/or connection ports, for example for operably connecting sensors, pulmonary delivery devices, and/or humidity control devices to the breathing circuit 126. In embodiments, the breathing circuit 126 may include one or more valves. For example, one or more one-way valves may be provided to direct gas flow in only one direction through the breathing circuit 126. Other valves may be included to open the breathing circuit 126 to the outside environment.

In embodiments, the elements of the breathing circuit 126 (hoses, tubes, masks, etc.) may be constructed using any material having the requisite chemical and mechanical properties. Example materials include but are not limited to biocompatible organic polymers such as ethyl vinyl acetate (EVA), high-density or low-density polyethylene (PE), and polypropylene (PP), polytetrafluoroethylene (PTFE), poly(tetrafluoroethylene-co-hexafluoropropylene) (FEP), modified ethylene-tetrafluoroethylene copolymer (ETFE), polyvinylidene fluoride (PVDF), polyethylene terephthalate polyester (PET-P), derivatives thereof, copolymers thereof, and blends thereof.

In embodiments, the breathing circuit 126 includes an upper airway device for delivering the mixed-gas inhalant 108 to the patient and receiving a carbon dioxide containing first mixed-gas exhalant 102 from the patient. The upper airway device may include a mask, an endotracheal tube, or other suitable device to deliver air to the upper airway. In at least some embodiments, the upper airway device includes a mask that provides a complete seal around the patient's mouth and nose. In one non-limiting example embodiment, the upper airway device may include a bubble-mask disposed about the head of the patient. In some embodiments, the upper airway device may include one or more valves or vents that open automatically in the event of system failure, such as during an electrical outage, to prevent suffocation. In other embodiments, a tent may be used to deliver the mixed-gas inhalant to the patient. In such embodiments, the patient may be placed within a tent forming a closed breathing environment, and the atmosphere within the tent is regulated in the same way as would be if using a mask-type upper airway device.

The patient's respiration provides a first mixed-gas exhalant 102 that includes carbon dioxide at a first concentration to the gas recirculation loop 112 via the at least one patient mixed-gas connection 116. Within the mixed-gas recirculation system 110, the first mixed-gas exhalant 102 initially passes through one or more heat and moisture exchange (HME) devices 170. Within the one or more HME devices 170, thermal energy and moisture from the first mixed-gas exhalant 102 is used to warm and humidify the inhalant contemporaneously passing through the one or more HME devices 170. In embodiments, the one or more HME devices 170 may include any number and/or combination of systems, components, and/or devices, capable of minimizing or even preventing drying of the respiratory mucosa of the patient. Although depicted as disposed internal to the mixed-gas recirculation system 110, in some embodiments, the one or more HME devices 170 may be disposed in, on, or about the breathing circuit 126 itself. In at least some embodiments, the one or more HME devices 170 may include one or more individually replaceable components within the mixed-gas recirculation system 110.

The one or more HME devices 170 assist in maintaining a proper moisture level in the mucous membranes in the airway. The one or more HME devices 170 assist in maintaining an adequate humidity level in the mixed-gas inhalant 108 provided to the patient, thereby minimizing patient discomfort and reducing the likelihood of infection due to the deterioration of the membrane barrier. The upper limit value and lower limit value for humidity in the mixed-gas inhalant 108 may be established for each patient. In at least some embodiments, the one or more HME devices 170 may maintain the humidity of the mixed-gas inhalant 108 within a range of from about 20% relative humidity to about 80% relative humidity.

The first mixed-gas exhalant 102 then passes through one or more first backflow prevention devices 164A. The one or more first backflow prevention devices 164A prevent the reverse flow of the first mixed-gas exhalant 102 to the patient via the breathing circuit 126. In embodiments, the one or more first backflow prevention devices 164A may include a check valve or similar device that closes in response to a reverse flow condition, thereby preventing the reverse flow from occurring. In at least some embodiments, the one or more first backflow prevention devices 164A may include one or more individually replaceable first backflow prevention devices 164A within the mixed-gas recirculation system 110.

The first mixed-gas exhalant 102, containing carbon dioxide at the first concentration exits the one or more first backflow prevention devices 164A and enters the one or more carbon dioxide removal devices 180. At least a portion of the carbon dioxide present in the first mixed-gas exhalant 102 is removed and retained by the one or more carbon dioxide removal devices 180 to provide a second mixed gas exhalant 104 discharge having carbon dioxide at a second concentration that is lower than the first concentration in the first exhalant 102. In some embodiments, such as depicted in FIG. 1, the one or more carbon dioxide removal devices 180 may be fluidly coupled to the one or more tee connections 142 such that the second exhalant 104 exits the one or more carbon dioxide removal devices 180 and mixes with a mixed-gas supply 106 to provide the first mixed-gas inhalant 108 prior to the one or more volumetric flow devices 150. In other embodiments, the one or more carbon dioxide removal devices 180 may be fluidly coupled to the one or more volumetric flow devices 150 and the mixed-gas supply 106 may also be fluidly coupled to the one or more volumetric flow devices 150 such that the second exhalant 104 and the mixed-gas supply 106 combine within the one or more volumetric flow devices 150 to provide the mixed-gas inhalant 108.

The one or more carbon dioxide removal devices 180 reduce the carbon dioxide concentration in the first mixed-gas exhalant 102 to provide the second mixed-gas exhalant 104 having carbon dioxide at a second concentration. In embodiments, the one or more carbon dioxide removal devices 180 may remove some or all of the carbon dioxide present in the first exhalant 102. For example, the one or more carbon dioxide removal devices 180 may remove about: 80% or more, 85% or more, 90% or more, 95% or more, 99% or more, or 99.9% or more of the carbon dioxide from the first exhalant 102. The second mixed-gas exhalant 104 contains carbon dioxide at a second concentration that is less than the first concentration in the first mixed-gas exhalant 102. In embodiments, the second mixed-gas exhalant 104 may have a second carbon dioxide concentration of about: 4% or less, 3% or less, 2% or less, 1% or less, 0.5% or less, 0.1% or less, or 0.05% or less of carbon dioxide by volume.

In embodiments, the one or more carbon dioxide removal devices 180 may include one or more chambers, reactor beds, or vessels containing one or more carbon dioxide sorbent materials. In embodiments, the one or more carbon dioxide removal devices 180 may include a non-reversible chemical fixation sorbent, such as sodium hydroxide, calcium hydroxide, lithium hydroxide, or potassium hydroxide that reacts with the carbon dioxide present in the first mixed-gas exhalant 102 to produce the corresponding carbonate and water. In embodiments, the one or more carbon dioxide removal devices 180 may include a non-re fixation sorbent such as a peroxide (e.g., sodium peroxide or lithium peroxide) capable of absorbing large amounts of carbon dioxide per unit weight. The use of such peroxides for carbon dioxide absorption provide the additional benefit of producing oxygen gas from the reaction with carbon dioxide.

In embodiments, the one or more carbon dioxide removal devices 180 may include a reversible chemical fixation sorbent, such as liquid amines. Liquid amines, such as monoethanolamine (MEA), are capable of binding carbon dioxide at low temperatures and releasing carbon dioxide upon thermal regeneration.

In embodiments, the one or more carbon dioxide removal devices 180 may include one or more membranes to separate the carbon dioxide from the first mixed-gas exhalant 102. In embodiments, one or more polymeric membranes function as a molecular sieve or may separate the gases based on solution diffusion. Membranes can contain pores of a controlled size relative to the kinetic (sieving) diameter of the gas molecule. The membranes can be made of organic or inorganic compounds. The membrane can be polymeric. The polymeric membrane can be porous and act as a molecular sieve, or non-porous and separate gases based on their solubility within the polymeric membrane as well as their physical-chemical interaction with the polymer. The polymeric membranes can be made out of suitable materials including but not limited to: cellulose acetate and derivatives thereof polysulfones, polyamides, polyvinyl acetate, and combinations of polymers, such as blends of polyimides. The polymeric membrane can be a mixed matrix membrane in which an organic polymer is combined with an inorganic compound to generate the membrane. In embodiments, the one or more carbon dioxide removal devices 180 may include one or more molecular sieves. The molecular sieve can contain silicate, silicone rubber, or zeolites, which are crystalline materials composed of silicon and aluminum (aluminosilicate). In embodiments, the one or more carbon dioxide removal devices 180 may include activated carbon, to absorb carbon dioxide from the first mixed-gas exhalant 102. In at least some embodiments, the one or more carbon dioxide removal devices 180 may include one or more individually replaceable carbon dioxide removal devices 180 within the mixed-gas recirculation system 110.

The second mixed-gas exhalant 104 exits the one or more carbon dioxide removal devices 180 and combines with the mixed-gas supply 106 to provide the mixed-gas inhalant 108. In embodiments, the second mixed-gas exhalant 104 and the mixed-gas supply 106 may be combined in one or more tee connections 142. In embodiments, the one or more tee connections 142 may include one or more internal structures such as fins or vanes to promote mixing of the second mixed-gas exhalant 104 with the mixed-gas supply 106. In at least some embodiments, the one or more tee connections 142 may include one or more individually replaceable tee connections 142 within the mixed-gas recirculation system 110.

The mixed-gas inhalant 108 flows from the one or more tee connections 142 to one or more volumetric flow devices 150, such as one or more breathing or respiratory bags. The one or more volumetric flow devices 150 retains the mixed-gas inhalant 108 until inhaled by the patient. In at least some embodiments, the one or more volumetric flow devices 150 may be disposed proximate a transparent panel disposed in, on, or about the external surface of the mixed-gas recirculation system 110. The transparent window beneficially permits the attendant or medical professional to visually ascertain the respiration volume of the patient. In embodiments, a scale may be formed in, on, or about the transparent window to beneficially permit the attendant or medical professional to estimate the volume of the mixed-gas inhalant 108 drawn by the patient. In at least some embodiments, the one or more volumetric flow devices 150 may include one or more individually replaceable volumetric flow devices 150 within the mixed-gas recirculation system 110.

In some embodiments, one or more relief valves may be fluidly coupled to the one or more volumetric flow devices 150. In such embodiments, the one or more relief valves may open, allowing escape of the mixed-gas inhalant 108 to the atmosphere when the pressure of the mixed-gas inhalant in the gas recirculation loop 112 is about: 5 cm H₂O or greater, 10 cm H₂O or greater, 15 cm H₂O or greater, 20 cm H₂O or greater, 25 cm H₂O or greater, or 30 cm H₂O or greater. In such embodiments, the one or more relief valves may actuate without requiring an external signal or a power supply.

The mixed-gas inhalant 108 is drawn from the one or more volumetric flow devices 150 as the patient inhales. The mixed-gas inhalant 108 flows through one or more first biological HEPA filters 140A. The one or more first biological HEPA filters 140A may include any number and/or combination of filter elements. For example, the one or more first biological HEPA filters 140A may include one or more particulate filter elements to minimize or eliminate the passage of particulates above a threshold diameter (e.g., 0.3 nanometers) and one or more biological filter elements to minimize or eliminate the passage of biological materials such as bacteria and viruses. The one or more first biological HEPA filters 140A may be arranged in parallel or in series within the gas recirculation loop 112. In embodiments, the one or more first biological HEPA filters 140A may prevent the propagation of particulate and/or biological contaminants through the gas recirculation loop 112. In at least some embodiments, the one or more first biological HEPA filters 140A may include one or more individually replaceable first biological HEPA filters 140A within the mixed-gas recirculation system 110.

The mixed-gas recirculation system 110 includes a plurality of instruments 144A-144n (collectively, “instruments 144”—two instruments, 144A and 144B are depicted in FIG. 1A although any number of instruments may be similarly positioned). As depicted in FIG. 1A, one or more second biological HEPA filters 140B may be used to prevent the transmission of particulate and/or biological contaminants from the gas recirculation loop 112 to at least some of the instruments 144B. Other instruments 144A may be directly coupled (i.e., without any intervening devices, components, or systems) to the gas recirculation loop 112. In embodiments, some or all of the instruments 144 may include single-use instruments that are replaced for each patient. In embodiments, some or all of the instruments may include multi-use instruments biologically isolated from the gas recirculation loop 112 and therefore are usable for multiple patients.

In embodiments, the instruments 144 may include any number and/or combination of powered or unpowered indicators and/or sensors. In embodiments, the instruments 144 may beneficially include only unpowered indicators thereby eliminating the need for an external power supply. In other embodiments, the instruments 144 may include powered sensors coupled to a renewable (e.g., solar cells or solar panel affixed to the mixed-gas recirculation system 110) and/or a rechargeable (e.g., secondary storage cells) power supply. The instruments 144 may be positioned within a particular component included in the mixed-gas recirculation system 110 or, in some cases, may be positioned proximate to an exterior to the component. For example, a volumetric flow rate instrument may be positioned within the inspiratory conduit, within the expiratory conduit, or both. In other embodiments, an analyte instrument to indicate the presence of an analyte within the first mixed-gas exhalant 102 may include an optical sensor positioned proximate an exterior surface of the expiratory conduit.

In embodiments, the instruments 144 may sense, indicate, and/or transmit any number and/or combination of parameters of the mixed-gas inhalant 108, the first mixed-gas exhalent 102, the second mixed-gas exhalant 104 or any combination thereof. Inhalant and/or exhalant parameters may include any number and/or combination of the following: linear flow rate, volumetric flow rate, flow patterns within and across breaths, total pressure, fluctuations in pressure within and outside the respiratory frequency range, hours of operation, hours of operation over a specified (e.g. 12-month) time period, time periods of operation of use over a specified time period (e.g. nightly operation over a 12-month time period), leak levels, change in pressure, change in flow of the chemical composition within the breathing circuit, breathing pattern and derivatives thereof, partial pressure, temperature, absolute humidity level, relative humidity level, and concentration. Instruments 144 that include sensors, such as pressure sensors are known in the art, including sensors for measuring the partial pressure of a particular gas. In embodiments, one or more instruments 144 may include a sensor for measuring partial pressure of oxygen gas may be a pO2 “Clark type” electrode.

In embodiments, the mixed-gas recirculation system 110 may include one or more instruments 144 to detecting the presence and/or degree of a gas leak from the gas recirculation loop 112. In embodiments, the mixed-gas recirculation system 110 may include one or more systems, devices, and/or components to controlling the delivery of the mixed-gas inhalant 108 to the patient to ensure that a sufficient amount of mixed-gas inhalant 108 is continuously administered, even in the presence of a gas leak.

In embodiments, the mixed-gas recirculation system 110 may include one or more acoustic sensors. The acoustic sensors may measure acoustic signals associated with different breathing conditions such as shallow breathing, wheezing, and/or normal breathing. The one or more instruments 144 may include one or more biometric sensors. In at least some embodiments, such biometric sensors may measure transcutaneous carbon dioxide. If the one or more instruments 144 indicate shallow breathing (such as determined by measurements from a spirometer) and/or acoustic vibrations that are consistent with wheezing in the breathing circuit, then one or more therapeutic agents may be introduced to the inhalant 108.

In embodiments, the mixed-gas recirculation system 110 includes one or more overpressure relief devices 160 and one or more vacuum relief devices 162. In embodiments, the one or more overpressure relief devices 160 may include an unpowered device such as a relief valve or Positive End Expiratory Pressure (PEEP) valve that opens to the surrounding environment to relive to atmosphere when the pressure in the mixed-gas recirculation system 110 increases above a defined value. For example, when the pressure in the mixed-gas recirculation system 110 increases above: 2 cm H₂O, 3 cm H₂O, 4 cm H₂O, 5 cm H₂O, or 6 cm H₂O. In embodiments, the one or more vacuum relief devices 162 may include an unpowered device such as a relief valve or Positive End Expiratory Pressure (PEEP) valve disposed to vent from the ambient environment to the mixed-gas recirculation system 110 when the pressure in the mixed-gas recirculation system 110 falls below a defined value. For example when the pressure in the mixed-gas recirculation system 110 falls below about: 2 cm H₂O, 1 cm H₂O, 0.5 cm H₂O, 0.25 cm H₂O, or 0 cm H₂O. In embodiments, either or both the one or more overpressure relief devices 160 and/or the vacuum relief devices 162 may be manually operated by an attendant or medical professional. In some embodiments, a pressure control device, such as an electronic pressure controller, or programmable controller circuitry may be configured to open the one or more pressure relief devices 160 and/or the one or more vacuum relief devices 162 based on one or more pressure measurements in the mixed-gas recirculation system 110.

The supply gas system 120 includes at least a first gas reservoir 122 to store a quantity of first gas and a second gas reservoir 124 to store a quantity of second gas. The mixed-gas recirculation system 110 may include any number of supply gas connections 114A-114n (collectively, “supply gas connections 114”—two 114A and 114B are depicted in FIG. 1 although any number of supply gas connections 114A-114n may be similarly positioned). In embodiments, the mixed-gas recirculation system 110 includes a first connection 114A to receive the first gas from the first gas reservoir 122 and a second connection 114B to receive the second gas from the second gas reservoir 124.

The supply gas system 120 provides the mixed-gas supply 106 to the gas recirculation loop 112. In embodiments, the mixed-gas supply 106 may be introduced to the second exhalant 104 via the one or more tee connections 142. In embodiments, the mixed-gas supply 106 may be introduced to the second exhalant 104 via the one or more volumetric flow devices 150 and/or the inspiratory conduit. The gas supply system 120 may include any number and/or combination of systems, devices, or components for controllably introducing the mixed-gas supply 106 into the gas recirculation loop 112.

In some embodiments, the supply gas system 120 may include but are not limited to: compressed or pressurized gas cylinders or bottles, liquefied gas sources, controlled intake of ambient air, and specialized bladders or bags. In other embodiments, the supply gas system 120 may include piped medical gas distribution systems found in hospitals and other medical facilities. In at least some embodiments, the supply gas system 120 may include one or more medical gas cylinders or bottles. Medical gas cylinders are available from a variety of suppliers. Medical gas cylinders are designed to meet U.S. Pharmacopeia (USP) and National Formulary (NF) standards and are manufactured in compliance with FDA current Good Manufacturing Practices (GMPs). Medical gas cylinders are typically made from high-quality steel, chromium-molybdenum allow, or aluminum, although any suitably strong and inert material may be used.

The supply gas system 120 may provide any number and/or combination of gases including but not limited to: oxygen, noble gases, biologically inert gases (e.g., N₂), or combinations thereof. In at least some embodiments, the supply gas system 120 may provide a mixture of oxygen and a noble gas, for example heliox—a mixture of oxygen and helium. In embodiments, the supply gas system 120 may provide one or more gases including but not limited to: nitrogen, argon, other inert gases, other noble gases, or any combination thereof. In some embodiments, the supply gas system 120 may provide one or more gases including: ambient air or medical grade air (78% Nitrogen and 21% oxygen with traces of water vapor, carbon dioxide, hydrogen, argon, and other components). In some embodiments, the supply gas system 120 may provide one or more gas mixtures including but not limited to: Nitrox, a mixture of nitrogen and oxygen; Heliox, a mixture of Helium and Oxygen; or Trimix, a mixture of Helium, Nitrogen, and Oxygen.

In at least some embodiments, the first gas reservoir 122 may store a first gas including oxygen and the second gas reservoir 124 may store a second gas that includes heliox. As used herein “heliox” refers to a gas mixture containing principally helium and oxygen with only low or trace levels of other gases or impurities. Commercially available standard heliox mixtures include from about 20% to about 30% oxygen and the remaining about 70% to about 80%, being helium. Heliox may include about: 10%-50%, 10%-40%, 20%-40%, or 20%-30% oxygen. In at least some embodiments, the heliox may include about 21% oxygen. Heliox may include about: 30%-90% helium, 50%-90% helium, or 70%-80% helium. In at least some embodiments, the heliox may include about 79% helium. The heliox supplies can come in standard pressurized gas cylinders containing, for instance, 25 ft³, 42 ft³, or 255 ft³ of heliox.

The pressure of the first gas 123 and the pressure of the second gas 125 are reduced using one or more pressure reducing devices 130A and 130B (collectively, “pressure reducing devices 130”), respectively. The pressure reducing devices 130 reduce the pressure of the first gas 123 and the second gas 125 to pressures suitable for use in the mixed-gas recirculation system 110. In at least some embodiments, the pressure reducing devices 130 may include any number and/or combination of pressure regulators, throttling valves, orifice plates, or similar pressure reducing components. In embodiments, the output pressure of each of the pressure reducing devices 130 may be manually adjusted by the attendant or medical professional. In other embodiments, the output pressure of each of the pressure reducing devices 130 may be automatically feedback or feed-forward controlled based on the pressure of the inhalant and/or exhalant using an electronic control device.

In embodiments, one or more flow control and gas blending devices 132 receive and combine the first gas 123 and the second gas 125 to provide a mixed-gas supply 106 having a prescribed first gas 123 to second gas 125 ratio. In embodiments, the one or more flow control and gas blending devices 132 may also include a plurality of flow control devices. In one example, as depicted in FIG. 1A, the one or more flow control and gas blending devices 132 may include a low-flow flowmeter output (“L”) and a high-flow flowmeter output (“H”). In embodiments, the flow rate of mixed-gas supply 106 through the one or more flow control and gas blending devices 132 and into the gas recirculation loop 112 may be manually or autonomously controlled. The one or more flow control and gas blending devices 132 may include any number and/or combination of systems, devices or combination of systems and devices suitable for mixing and/or controlling the flow of the mixed-gas supply 106 to the gas recirculation loop 112.

Although not depicted in FIG. 1A, in at least some embodiments, the mixed-gas recirculation system 110 may include one or more gas cooling devices (also referred to herein as “gas cooler”) disposed in the gas recirculation loop 112. Warm mixed-gas exhalant 102 may impact gas properties qualities, such as relative humidity. Additionally, in at least some embodiments, the removal of carbon dioxide from the first mixed-gas exhalant 102 by the one or more carbon dioxide removal devices 180 may involve exothermic reactions which generate heat, which further increases the temperature of the second mixed-gas exhalant 104 within the gas recirculation loop 112. Mixed-gas inhalant 108 supplied at too high a temperature may lead to dehydration of the sensitive lining of the patient's respiratory system (i.e. the mucous membranes) and lead to bronchospasms, respiratory distress, or even burns of the respiratory system lining.

In at least some embodiments, the mixed-gas recirculation system 110 may include a gas cooling device that reduces the temperature of the mixed-gas inhalant 108. In embodiments, such gas cooling devices may be disposed downstream of the one or more carbon dioxide removal devices 180 to control the temperature of the second mixed-gas exhalant 104 and/or the mixed-gas inhalant 108, thereby increasing patient safety and comfort. In such embodiments all or a portion of the second mixed-gas exhalant 104 and/or the mixed-gas inhalant 108 may be passed through the one or more gas cooling devices.

Although not depicted in FIG. 1A, in some embodiments, the mixed-gas recirculation system 110 may also include one or more therapeutic or diagnostic agents administered to the patient via the mixed-gas inhalant 108. The one or more therapeutic or diagnostic agents may include any suitable agent that can be aerosolized and carried by the mixed-gas inhalant 108 to the patient. Exemplary therapeutic agents include but are not limited to bronchodilators, such as beta mimetics, anticholinergics, anti-inflammatory agents, steroids, and mast cell stabilizers. Exemplary biological agents include but are not limited to proteins, peptides, cells, and other biological agents. In embodiments, the one or more therapeutic or diagnostic agents may be administered continuously or intermittently, in any desired pattern. In some embodiments, the one or more therapeutic or diagnostic agents may be administered via a humidifier. For example, an ultrasonic humidifier may be fluidly coupled to the gas recirculation loop 112 to ensure proper humidity levels for mixed-gas inhalant 108.

The mixed-gas recirculation system 110 may be used to administer one or more therapeutic agents to a patient while the patient sleeps. During a normal sleep period, humans experience a change in bronchial tone over time, which follows a standard circadian rhythm. Typically, the worst bronchial tone is present in the middle of the night, such as around 2-5 am, typically around 4 am. The amount of time since the last dose of a bronchodilator also impacts bronchial tone, with decreases in bronchial tone over time, such as over a period of 4 to 6 hours since the previous dose.

The presence of a noble gas, such as helium, in the system can facilitate delivery of the one or more therapeutic agents to the deep lung. Additionally, small particles (e.g. less than 5 microns in diameter) containing the one or more therapeutic agents may be stabilized by helium. See, O'Callaghan, et al., “The Effects of Heliox on the Output and Particle-Size Distribution of Salbutamol Using Jet and Vibrating Mesh Nebulizers”, Journal of Aerosol Medicine, 20(4): 434-444 (December 2007).

The mixed-gas recirculation system 110 may include sensors for measuring various parameters in the gas recirculation loop 112, for measuring various parameters in the ambient environment external to the mixed-gas recirculation system 110, and/or for measuring various patient biometric parameters. A variety of microsensors and microelectromechanical sensors (MEMS)-based sensors are commercially available, including MEMS biosensors for detecting specific chemical or biological pathogens, MEMS pressure or temperature sensors, etc.

The mixed-gas recirculation system 110 may include one or more sensors for measuring parameters external to the mixed-gas recirculation system 110, including parameters associated with the ambient environment about the mixed-gas recirculation system 110. The mixed-gas recirculation system 110 may also include sensors for measuring ambient parameters external to the mixed-gas recirculation system 110. Such ambient environmental sensors may include but are not limited to: temperature sensors, humidity sensors, power sensors, etc. For example, a humidity sensor may detect the relative humidity of the ambient air external to the mixed-gas recirculation system 110. In embodiments, a power sensor can detect the presence or absence of an external power supply, allowing the controller to respond to a loss of power.

The mixed-gas recirculation system 110 may include sensors for measuring biometric parameters associated with the patient. Exemplary biometric sensors include heart rate monitors, grip pressure sensors, cameras, blood pressure monitors, moisture sensors, breathing sensors, brain sensors, etc. In embodiments, the sensors can be a fingertip sensor or wristband worn by the user that measures pulse rate or CO2 levels in the blood. In other embodiments, the sensors can be sound sensors or chest bands measuring respiration rate.

Referring next to FIG. 1B, in at least some embodiments, the mixed-gas recirculation system 100 may include system control circuitry 190. In such embodiments, the control circuitry 190 may include a user interface 192 and a suitable device for processing information collected using one or more system, ambient environment, and/or patient biometric sensors 144. The user interface 192 may include one or more input devices such as one or more keypads, buttons, toggles, and/or switches. In embodiments, the user interface 192 may include one or more output devices, such as one or more audio output devices, one or more haptic output devices, one or more visual output devices, or combinations thereof. The one or more visual output devices may include but are not limited to: a light emitting diode (LED) display, organic LED (OLED) display, an active matrix OLED display, a graphical LCD (GLCD), a thin film transistor LCD (TFTLCD), a super TFT LCD, a 7-segment LCD, an in-plane switching LCD (IPSLCD), LED backlit IPS TFT LCD display, a holographic display, a 3-dimensional display, a plasma display, or a combination thereof. In some embodiments, the one or more visual output devices may include one or more touch-screen interfaces. The user interface 192 may be electronically coupled to the control circuitry 190. Additionally, or alternatively, the user interface 192 may communicate wirelessly with the control circuitry via one or more wireless signals such as radio frequency (RF), IEEE 802.15 (e.g., BLUETOOTH®, ZigBee, MiWi, Body Area Network—latest version) protocol, near field communication (NFC) protocol, a local area network (LAN), a wireless local area network (WLAN), a personal area network (PAN), an optical signal, or other suitable wireless signal. The control circuitry 190 may communicatively coupled to one or more local or remote data storage devices including but not limited to: one or more non-transitory storage devices, one or more volatile storage devices such as random access memory circuitry, a read-only memory, and/or an erasable programmable read-only memory (EPROM or Flash memory).

The control circuitry 190 may include single- or multi-core processor circuitry suitable for processing information and/or data communicated by one or more indicators/sensors 144 including but not limited to: a microprocessor circuitry to execute instructions or an application implemented in hardware, software, firmware, or a combination thereof. The instructions or applications may be implemented in microprocessor hardware using any of or a combination of the following technologies, which are well known in the art, including, but not limited to: a discrete logic circuit(s) having logic gates for implementing logic functions upon data signals, an application specific integrated circuit (ASIC) having appropriate combinational logic gates, a programmable gate array(s) (PGA), or a field programmable gate array (FGPA). The processing device can also include a port for connecting to a data storage device such as, but not limited to, a portable computer diskette, a random access memory, a read-only memory, an erasable programmable read-only memory (EPROM or Flash memory), a secured digital (SD) card, or a portable compact disc read-only memory.

In embodiments, the control circuitry 190 may include transmitter circuitry for unidirectionally or bidirectionally communicating one or more signals to other components included in the mixed-gas recirculation system 110, such as the pressure reducing devices 130 and/or the gas blending device 132. The one or more signals may be electrical, optical, or wireless. The control circuitry 190 may be connected to other components of the mixed-gas recirculation system 110 by a physical connection, such as a wire or cable; an optical connection, such as an optical fiber; or a wireless connection, such as by radio frequency (RF), IEEE 802.15 (e.g., BLUETOOTH®, ZigBee, MiWi, Body Area Network—latest version), near field communication (NFC®) protocol, a local area network (LAN), a wireless local area network (WLAN), a personal area network (PAN), an optical signal, or other suitable wireless signal.

In embodiments, the control circuitry 190 may generate one or more alarms. Alarms may be tactile, audible, visual, or electronically transmitted alarms or warnings, for example for alerting the patient or for alerting a health-care provider. Alarms can include alarms for low pressure, high pressure, fire, power outage, power surge, diminishing gas supplies, system malfunction, system service, etc. In embodiments, the control circuitry 190 may include one or more timers, for instance for tracking the time the system has been operating, for tracking a pre-determined shutoff time, for tracking the time since last service, for tracking the time since the gas supplies were changed, etc. The timers can be visually displayed, or in some cases are used to trigger one of the alarms described above or to shut off the system.

Heliox treatments have been used to treat various diseases and symptoms that afflict the airway, including, but not limited to COPD, asthma, upper airways obstruction, and croup. See, Reuben and Harris, Emerg Med J, 21: 131-135 (2004). The predetermined ranges reflect differences in treatment and symptoms experienced by a patient and by physiological characteristics of the patient. Insofar as heliox contains helium and oxygen, and the ratio of the two components is often important to achieving a desired effect, preferably predetermined ranges are set for both helium and oxygen, or any other supplied gas.

The upper limit value and lower limit value for the predetermined range for helium can be any suitable value. In embodiments, the upper limit value and lower limit values of the predetermined range are set to keep helium concentration within the gas recirculation loop 112 at any concentration or concentrations between 50 and 79%. Insofar as there are primarily only two sources of gases being mixed (i.e., a noble gas/oxygen mixture and oxygen), the concentration, partial pressure helium within in the circuit can be indirectly determined based on the concentration, partial pressure, or volume of oxygen, which is constantly directly measured. In embodiments, indirect determination of helium amount is a feasible and cost-effective way to measure helium concentrations within the gas recirculation loop 112. In some embodiments, the noble gas concentration in the mixed-gas inhalant 108 may be directly measured using a sensor.

The predetermined ranges may be adjusted accordingly based on the type of input received. Alternatively, the control circuitry 190 may be configured to convert the units of input into another unit type to be used with a predetermined range. For example, if the input is the partial pressure of oxygen, and the predetermined helium range is based on the concentration of oxygen, the control circuitry 190 may be configured to convert the partial pressure of oxygen input into a concentration of oxygen value to be used to indirectly determine if the amount of helium within the gas recirculation loop 112 is correct.

The upper limit value and lower limit value for the predetermined range for oxygen can be any suitable value. In embodiments, the upper limit value and lower limit values of the predetermined range are set to keep oxygen concentration within the mixed-gas inhalant 108 at any concentration or concentrations between 21 and 50%. As one having ordinary skill will appreciate, any measure of the amount of oxygen may be used, such as, but not limited to concentration, partial pressure, or volume. The predetermined ranges may be then adjusted accordingly based on the type of input received. Alternatively, the control circuitry 190 may be configured to convert the units of input into another unit type to be used with a predetermined range. For example, if the input is the partial pressure of oxygen and the predetermined range is based on the concentration of oxygen, the control circuitry 190 may be configured to convert the partial pressure input into a concentration value to be used to determine if the amount of oxygen in the mixed-gas inhalant 108 is correct.

Carbon dioxide is introduced to the mixed-gas recirculation system 110 by the patient. The amount of carbon dioxide (e.g. concentration or partial pressure) within the breathing circuit may be continuously measured. Such continuous purpose serves two purposes: (1) as a safety monitor to prevent dangerous build of carbon dioxide breathing circuit and (2) to monitor the level of functionality of the carbon dioxide removal device 180. The upper limit value and lower limit value for the predetermined range for carbon dioxide can be any value from negative infinity to positive infinity. In embodiments, the upper limit value and lower limit values of the predetermined range may be set to keep partial pressure of carbon dioxide within the gas recirculation loop 112 at any partial pressure not to exceed 30,000 parts per million (ppm) for more than fifteen minutes and not to exceed 10,000 ppm over an 8-hour period. As one having ordinary skill will appreciate, any measure of the amount of carbon dioxide may be used, such as, but not limited to concentration, partial pressure or volume. The predetermined ranges may be then adjusted accordingly based on the type of input received. Alternatively, the control circuitry may be configured to convert the units of input into another unit type to be used with a predetermined range. For example, if the input is the partial pressure of oxygen and the predetermined range is based on the concentration of oxygen, the control circuitry may be configured to convert the partial pressure input into a concentration value to be used to determine if the amount of oxygen in the mixed-gas inhalant 108 is correct.

The mixed-gas recirculation system 110 relies upon a delivering noble or inert gas and oxygen mixture under low pressure within the breathing circuit 126. Thus, preferably the total air pressure within the breathing circuit 126 is less than 4 cm H₂O (approximately 0.392 kPa). Preferably, the upper limit and lower limit of the predetermined range is set such that the air pressure within the breathing circuit 126 is less than 4 cm H₂O but greater than approximately 2 cm H₂O.

In embodiments, if the quantity of noble or inert gas present in the gas recirculation loop 112 is outside the predetermined range set for the noble or inert gas, the one or more flow control and gas blending devices 132 may be manually or autonomously increase or decrease the noble or inert gas in the mixed-gas supply 106 such that the noble or inert gas present in the gas recirculation loop 112 returns to an acceptable value. In embodiments, if the amount of oxygen present in the gas recirculation loop 112 is outside the predetermined range set for the oxygen, the one or more flow control and gas blending devices 132 may be manually (by the patient, an attendant, or a medical professional) or autonomously (via control circuitry 190) increase or decrease the oxygen in the mixed-gas supply 106 such that the oxygen present in the gas recirculation loop 112 returns to an acceptable value.

In embodiments, in the event that the carbon dioxide concentration present in the gas recirculation loop 112 exceeds the upper limit of a predetermined carbon dioxide range, an additional carbon dioxide removal device 180 may be fluidly coupled to the gas recirculation loop 112 to remove excess carbon dioxide from the breathing circuit 108. In other embodiments, one or more valves fluidly coupled to the gas recirculation loop 112 may allow at least a portion of the mixed-gas exhalant 102 and/or the mixed-gas inhalant 108 to escape to the ambient environment.

Excess carbon dioxide must be avoided in the gas recirculation loop 112 because of unwanted and life threatening effects of high levels of carbon dioxide exceeding 30,000 parts per million (ppm) in any consecutive 15-minute time period or 10,000 ppm over an 8-hour time period. When properly functioning, the amount of carbon dioxide within the gas recirculation loop 112 is maintained below approximately 10,000 ppm by the one or more carbon dioxide removal devices 180. However, in the event the one or more carbon dioxide removal devices 180 are insufficient to keep the amount of carbon dioxide below the 10,000 ppm threshold one or more valves fluidly coupled to the gas recirculation loop 112 may release at least a portion of the mixed-gas exhalant 102 and/or the mixed-gas inhalant 108 to the surrounding atmosphere.

In some embodiments, one or more biometric sensors may be used to detect or monitor one or more biometric parameters associated with the patient. In some embodiments, the control circuitry 190 may alter or adjust one or more components included in the mixed-gas recirculation system 110. For example, a pulse oximeter may be used to sense the patient's blood oxygen concentration. If the measured blood oxygen concentration is lower than the lower limit value of the predetermined range, the oxygen level in the mixed-gas supply 106 may be increased to increase the oxygen concentration in the mixed-gas inhalant 108. increase the influx of oxygen into the breathing circuit 108.

In one embodiment, the mixed-gas recirculation system 110 can be used to adjust the core temperature of a patient's body, such as by delivering a reduced temperature mixed-gas inhalant 108 to the patient. Modifying a patient's core body temperature can facilitate sleep or ameliorate the body's inflammatory response. The patient's core body temperature may be measured using one or more temperature sensors. For example, biometric sensors on the patient can be used to indicate a patient's core body temperature information an attendant, medical professional, or control circuitry. Additionally, or alternatively, the system may include a temperature sensor in the expiratory circuit, which measures the temperature of the first mixed-gas exhalant 102 as they exit the breathing circuit 126. The temperature of the mixed-gas inhalant 108 may be modified in any desired manner. For example, the temperature may be varied in correspondence with the patient's circadian rhythm.

In some embodiments, the mixed-gas recirculation system 110 may include a spirometer for measuring the flow of mixed-gas inhalant 108 to the patient. The spirometer may be an in-line device that is integrated with the breathing circuit 126. Exemplary spirometers include pneumotachographs, turbine spirometers, and ultrasound spirometers. Suitable spirometers are known to those of ordinary skill in the art.

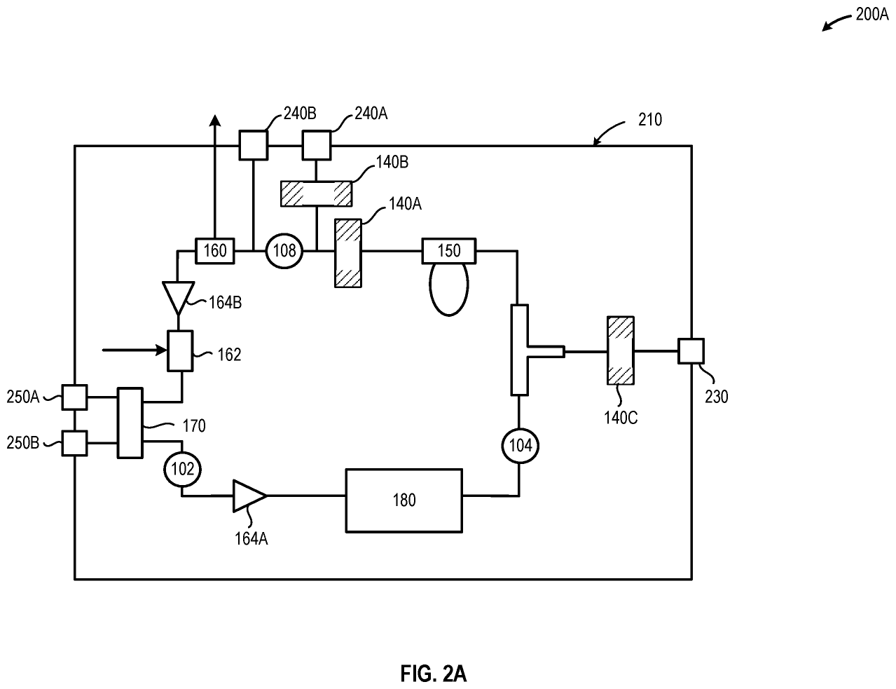

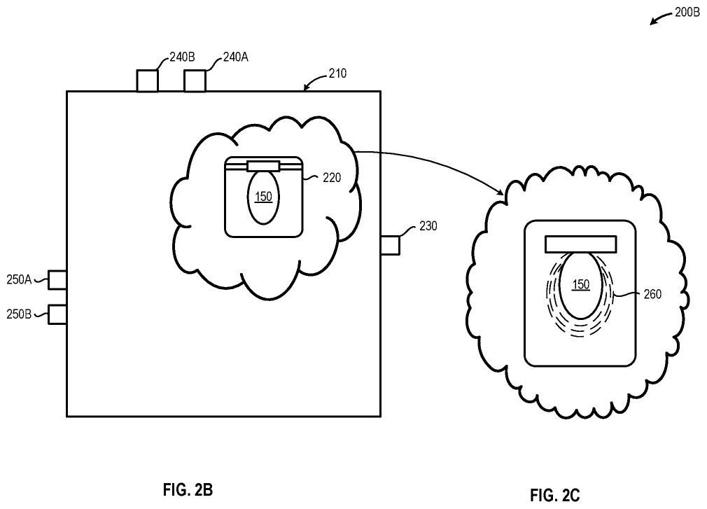

FIG. 2A is a schematic diagram of a replaceable first module 200 that includes the gas recirculation loop 112 in which the components depicted are replaced for each new patient, in accordance with at least one embodiment described herein. FIG. 2B is an external elevation view of the replaceable first module 200 depicted in FIG. 2A, in accordance with at least one embodiment described herein. FIG. 2C is a detail view of the illustrative transparent panel 220 that allows visual confirmation of inflation of the one or more volumetric flow devices 150, in accordance with at least one embodiment described herein.

Referring collectively to FIGS. 2A-2C, The heat and moisture exchange device 170 may be fluidly coupled to a mixed-gas inhalant connection 250A and a first mixed-gas exhalant connection 250B, both disposed in a location readily accessible to the patient, an attendant, or a medical professional. For example, the mixed-gas inhalant connection 250A and the first mixed-gas exhalant connection 250B may be disposed on an external surface of the first module housing 210. In some embodiments, the mixed-gas inhalant connection 250A and the first mixed-gas exhalant connection 250B may be combined to provide a single breathing circuit connection 250.

The first module 200 includes at least one mixed-gas supply connection 230 disposed in a location readily accessible to the patient, an attendant, or a medical professional. For example, the mixed-gas supply connection 230 may be disposed on an external surface of the first module housing 210. In at least some embodiments, the mixed-gas supply connection 230 may include a quick-connect or similar type fitting. In some embodiments, the mixed-gas supply connection 230 may include a threaded connection or a camlock type fitting. The mixed-gas supply connection 230 may be fluidly coupled to the third biological HEPA filter 140C.

The first module 200 also includes any number of instrument, indicator, or sensor connections 240A-240n (collectively, “instrument connections 240”—in FIGS. 2A-2C, only two such connections 240A, 240B are shown). In some embodiments, at least a portion of the instrument connections 240, such as instrument connection 240A in FIGS. 2A-2C, may be directly connected to the gas recirculation loop 112. In some embodiments, at least a portion of the instrument connections 240, such as instrument connection 240B in FIGS. 2A-2C, may be fluidly coupled to a second biological HEPA filter 140B to isolate the connected instrument from particulate and/or biological contaminants present in the gas recirculation loop 112. In some instances the instrument connections 240 may include a quick-connect or similar type fittings. In some implementations, the instrument connections 240 may include one or more male or female, single- or multi-conductor connectors.

As depicted in FIGS. 2B and 2C, in embodiments, the housing 210 may include one or more transparent panels 220 to permit the patient, attendant, or medical professional to visually observe the operation of one or more components that are disposed in the housing 210, such as the volumetric flow indicator 150, while the mixed-gas recirculation system 100 is in operation. In at least some implementations, the transparent panel 220 may include one or more scales or similar visual markings 260 capable of permitting the patient, attendant, or medical professional to estimate the mixed-gas inhalant 108 flow volume through the volumetric flow indicator 150. In some instances, the one or more transparent panels 220 may be formed from one or more thermally or chemically cured plastic materials such as polycarbonate. In other instances, the one or more transparent panels 220 may be formed using safety glass or similar shatterproof glass materials (e.g., Al₂O₃—sapphire glass, Gorilla Glass®, or similar).

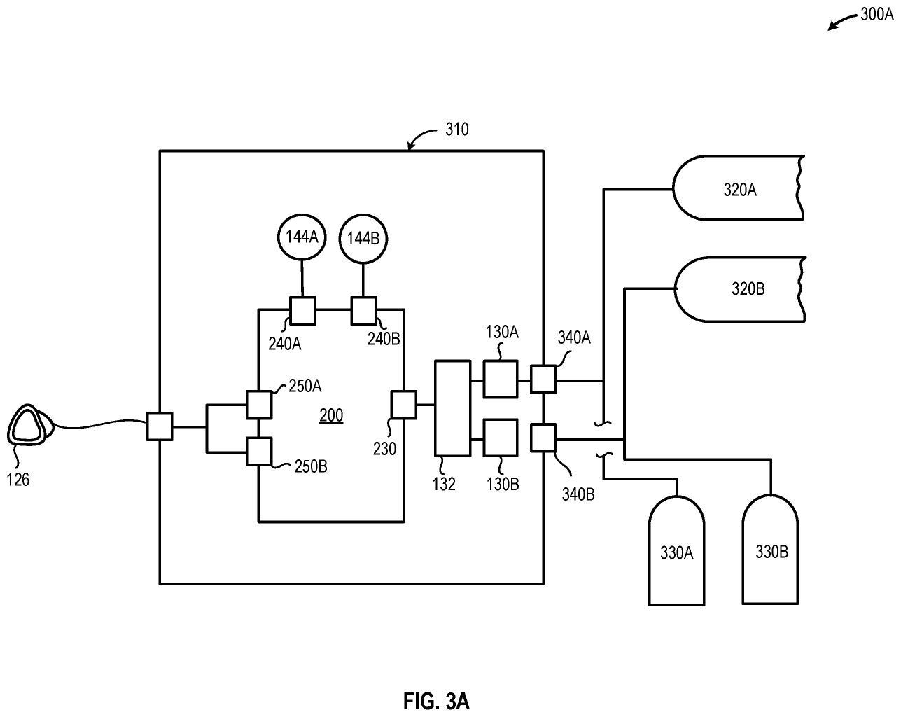

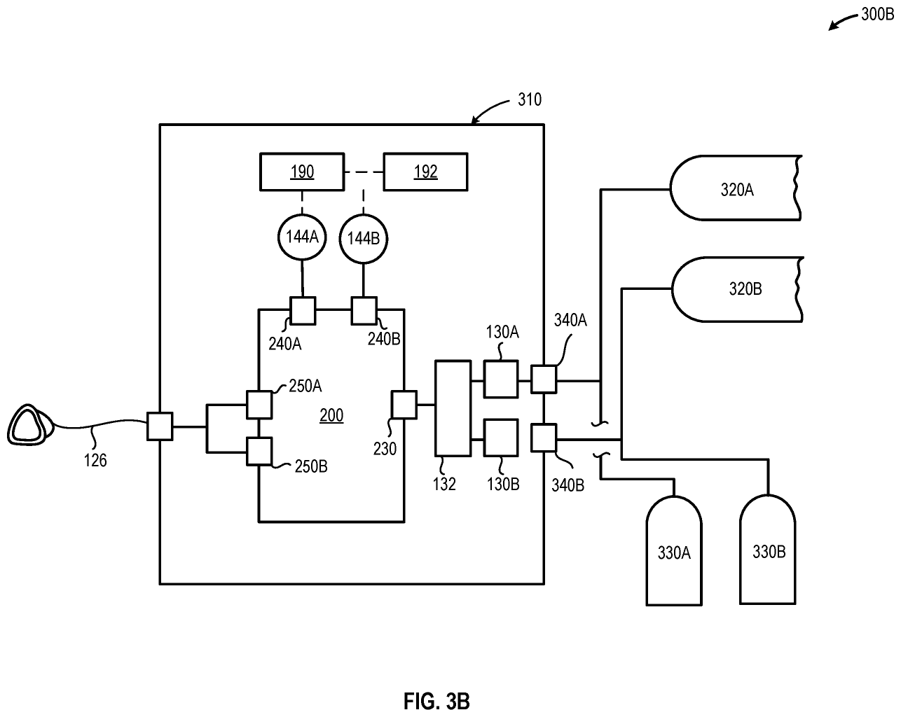

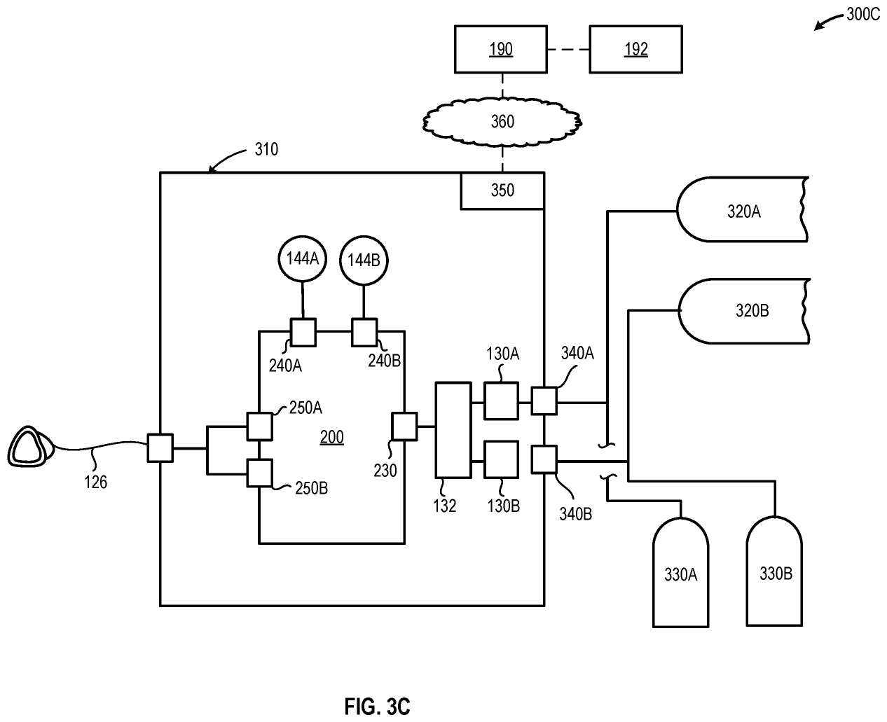

FIG. 3A is a schematic diagram of a system 300A in which the single-use or replaceable first module 200 depicted in FIGS. 2A-2C operably couples to a multi-use second module 310 that includes the instruments 144A, 144B, the gas blending and flow control devices 132, and the one or more pressure reducing devices 130A, 130B to manually and/or pneumatically control the operation of the gas recirculation loop 112, in accordance with at least one embodiment described herein. FIG. 3B is a schematic diagram of another system 300B in which the second module 310 includes local control circuitry 190 and a local user interface 192 to electronically control the operation of at least a portion of the gas recirculation loop 112, in accordance with at least one embodiment described herein. FIG. 3C is a schematic diagram of yet another system 300C in which the second module 310 includes network connected remote control circuitry 190 and a remote user interface 192 to electronically control the operation of at least a portion of the gas recirculation loop 112, in accordance with at least one embodiment described herein.

As depicted in FIGS. 3A-3C, in embodiments, the mixed-gas recirculation system 300A-300C includes a single-use gas recirculation loop first module 200 that includes relatively low-cost single-use components that may be contaminated with biological materials operably coupled to a multi-use second module 310 that includes relatively high-cost multi-use components isolated from contamination by particulates and/or biological materials, for example using biological HEPA filters 140. The second module 310 may include a housing in which one or more instruments 144, the gas blending and flow control devices 132, and the one or more pressure reducing devices 130A, 130B are at least partially disposed.

The second module 310 includes a first supply gas connection 340A and a second supply gas connection 340B for the fitment or connection of one or more external storage devices such as one or more first gas bulk storage reservoirs 320A and one or more second gas bulk storage reservoirs 320B, such as found in a hospital or other similar institutional settings. In other embodiments the first supply gas connection 340A and the second supply gas connection 340B may be used for the fitment or connection of one or more smaller, or portable, first gas storage tanks 330A and one or more smaller, or portable, second gas storage tanks 330B, such as found in residential or commercial facilities such as nursing homes. The second module 310 also includes one or more ports 350 to fluidly couple a breathing circuit 126 to the gas recirculation loop 112.

FIG. 3B depicts a second module 310 that includes local control circuitry 190 and a local user interface 192 to receive information and/or data from one or more instruments coupled to the gas recirculation loop, one or more instruments disposed in an external environment about the gas recirculation system 300B, and/or one or more instrument operably coupled to the patient. In addition, the local control circuitry 190 may generate one or more outputs to control the operation of the gas blending and flow control devices 132, the one or more pressure reducing devices 130A, 130B, the one or more carbon dioxide removal devices 180, or any combination thereof.

FIG. 3C depicts a second module 310 that includes remote control circuitry 190 and a remote user interface 192 coupled via one or more networks 360 to a transceiver 350 operably coupled to the mixed-gas recirculation system 300C to receive information and/or data from one or more instruments coupled to the gas recirculation loop, one or more instruments disposed in an external environment about the gas recirculation system 300B, and/or one or more instrument operably coupled to the patient. In addition, the remote control circuitry 190 may generate one or more outputs to control the operation of the gas blending and flow control devices 132, the one or more pressure reducing devices 130A, 130B, the one or more carbon dioxide removal devices 180, or any combination thereof.

The one or more networks 360 may include any number and/or combination of local area networks (LANs), wireless local are networks (WLANs), metropolitan area networks (MANs), worldwide area networks (WWAN—e.g., the Internet). The remote control circuitry 190 may include a network server, a cloud-based server, or similar. Beneficially, a single such remote control circuitry 190 may monitor and/or control any number of mixed-gas recirculation systems 300C.

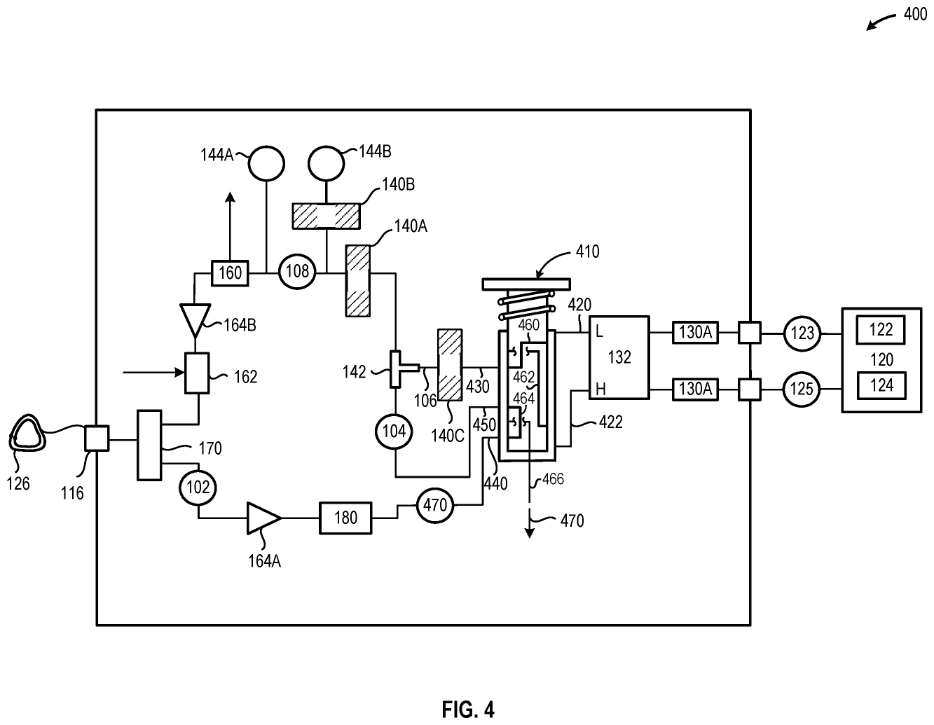

FIG. 4 is a schematic diagram of an illustrative mixed-gas delivery system 400 similar to that depicted in FIG. 1A, in which a purge valve 410 has been installed in the gas recirculation loop 112 and in which the flow control device 132 now includes a low-flow mixed-gas supply connection 420 and a high-flow mixed-gas supply connection 422, in accordance with at least one embodiment described herein. The purge valve 410 is manually or autonomously, reversibly positioned in either of: a first position associated with a RUN mode in which the gas recirculation loop 112 remains closed or a second position associated with a PURGE mode in which the gas recirculation loop 112 is open. The system 400 replaces nitrogen gas present in the ambient environment with helium. In order to replace the nitrogen, the gas stored in the system 400, in the patient's lungs, and to a lesser extent, the gas in the patient's circulatory system and tissues may be replaced or purged from the gas recirculation loop 112. The purge valve 410 permits the controlled release of gas from the gas recirculation loop 112 to the ambient atmosphere. The release of the gas to the ambient atmosphere permits the gas recirculation loop 112 to maintain a defined heliox gas ratio for the prescribed therapy. When gas ratio stasis is achieved within the gas recirculation loop 112, the purge valve 410 returns to the first position (i.e., the RUN mode).

The purge valve 410 is fluidly coupled to the gas recirculation loop 112. When in the first (i.e., RUN) position, gas from the gas recirculation loop 112 enters the purge valve 410 at 440, flows through the purge valve conduit 464 and exits the purge valve 410 at 450. While the purge valve 410 remains in the first position, mixed-gas provided by the supply gas system 120 flows from the low-flow mixed-gas supply connection 420 of the flow control device 132 into the purge valve 410 through conduit 460 and exits the purge valve 410 at 430. The low-flow mixed-gas supply 106 then flows through the third HEPA filter 140C and into the mixing tee 142. During normal operation gas flows through the gas recirculation loop 112 through the patient, and back to the gas recirculation loop 112.

During an initial patient setup, a healthcare provider (HCP) will position the purge valve 410 in the second (i.e., PURGE) position. In at least some embodiments, the purge valve 410 may include a spring loaded valve biased to the first (i.e., RUN) position. When the purge valve 410 is placed in the second position by the HCP, mixed-gas provided by the supply gas system 120 flows from the high-flow mixed-gas supply connection 422 of the flow control device 132 into the purge valve 410 through conduit 462 and exits the purge valve 410 at 430. With the purge valve 420 in the second position, the gas recirculation loop 112 fluidly couples to the purge conduit 466 and a purge gas 470 flows from the purge valve 410 to the ambient atmosphere. The high-flow mixed-gas supply 106 then flows through the third HEPA filter 140C, into the mixing tee 142, then into the gas recirculation loop 112. During the purge, the HCP will monitor the system gas ratio using a gas composition device 470. When the gas composition device 470 indicates a minimum amount of nitrogen is measured in the gas recirculation loop 112, the HCP will return the purge valve 410 to the first position. In some embodiments, the gas composition device 470 may include one or more devices to measure a quantity of helium present in the gas recirculation loop 112, and when the helium reaches the prescribed level, the system is returned to normal operation.

In embodiments a low-flow flowmeter and a high-flow flowmeter may be coupled in parallel to the flow control device 132, with each of the flowmeters sourcing mixed supply gas from their respective sources. When the purge valve 410 is positioned in the second (i.e., PURGE) position gas flows through the gas recirculation loop 112 and/or the high-flow flowmeter may be opened top permit additional gas to flow through the gas recirculation loop 112. The low-flow flowmeter will not allow back flow due to the design of the flow control device 132. The exhaled gas from the patient is vented to the atmosphere by disconnecting the expiratory limb of the breathing circuit 126. Either the HCP can time the length of the purge per pre-determined calculations, or monitor the gas composition device. When the purge is completed, the high-flow flowmeter is turned off and the expiratory limb is reconnected to the breathing circuit 126.

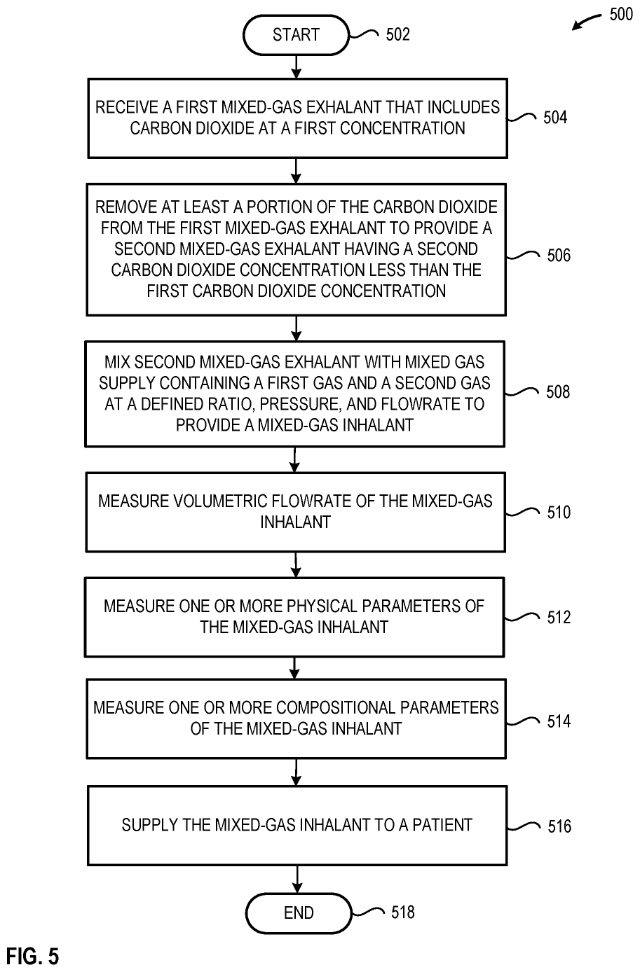

FIG. 5 is a high-level flow diagram of an illustrative mixed-gas recirculation method 500, in accordance with at least one embodiment described herein. The method 500 employs a gas recirculation loop that may be pneumatically operated, beneficially minimizing or even eliminating the need for an external power supply. The mixed-gas supply system disclosed herein may be operated by the patient, an attendant, or medical professional. When the ease of operation is combined with no need for a power source, the systems and methods disclosed herein may be used in unimproved locations or in locations where power supplies have been disrupted (e.g., areas impacted by natural disasters). The method 500 commences at 502.

At 504, a first mixed-gas exhalant that includes carbon dioxide at a first concentration is received at a first connection to a gas recirculation loop.

At 506, a carbon dioxide removal device disposed in the gas recirculation loop removes at least a portion of the carbon dioxide from the first mixed-gas exhalant to provide a second mixed-gas exhalant that includes carbon dioxide at a second concentration less than the first concentration.

At 508, the second mixed-gas exhalant combines with a mixed-gas supply that includes a first gas and a second gas to provide a mixed-gas inhalant at a volumetric flow rate within a defined range.

At 510, a volumetric flow device measures the volumetric flow rate of the mixed-gas inhalant through the gas recirculation loop.

At 512, a first instrument measures one or more physical parameters of the mixed-gas inhalant.

At 514, a second instrument measures one or more compositional parameters of the mixed-gas inhalant.

At 516, the mixed-gas inhalant is supplied to the patient via a second connection. The method 500 concludes at 518.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

As used in any embodiment herein, the terms “system” or “module” may refer to, for example, software, firmware and/or circuitry configured to perform any of the aforementioned operations. Software may be embodied as a software package, code, instructions, instruction sets and/or data recorded on non-transitory computer readable storage mediums. Firmware may be embodied as code, instructions or instruction sets and/or data that are hard-coded (e.g., nonvolatile) in memory devices.

As used in any embodiment herein, the term “circuitry” may comprise, for example, singly or in any combination, hardwired circuitry, programmable circuitry such as computer processors comprising one or more individual instruction processing cores, state machine circuitry, and/or firmware that stores instructions executed by programmable circuitry or future computing paradigms including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more mediums (e.g., non-transitory storage mediums) having stored therein, individually or in combination, instructions that when executed by one or more processors perform the methods. Here, the processor may include, for example, a server CPU, a mobile device CPU, and/or other programmable circuitry. Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software executed by a programmable control device.

Thus, the present disclosure is directed to systems and methods of providing a mixed-gas inhalant to a patient via a gas recirculation loop. The gas recirculation loop receives a first mixed-gas exhalant having a first carbon dioxide concentration from the patient, one or more carbon dioxide removal devices discharge a second mixed-gas exhalant having a second carbon dioxide concentration that is less than the first carbon dioxide concentration. The second mixed-gas exhalant is combined with a mixed-gas supply to provide a mixed-gas inhalant. The mied-gas supply includes a first gas and a second gas. The mixed-gas supply is pressure and flow controlled to produce a mixed-gas inhalant having a defined composition delivered to the patient at a defined volumetric flow rate. The first gas may include a gas containing oxygen and the second gas may include a gas mixture containing a noble or inert gas and oxygen.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

According to example 1, there is provided a mixed-gas recirculation system. The mixed-gas recirculation system may include: a gas recirculation loop that includes: a first connection to receive a first mixed-gas exhalant from a patient, the first mixed-gas exhalant having carbon dioxide at a first concentration; a carbon dioxide removal device to remove at least a portion of the carbon dioxide present in the first exhalant received from the patient to provide a second mixed-gas exhalant having carbon dioxide at a second volumetric concentration less than the first volumetric concentration; a mixer to combine the second exhalant with volumetrically controlled flowrate of a mixed-gas supply that includes a first gas and a second gas to provide a mixed-gas inhalant having a volumetric feed rate within a defined range; an overpressure protection device fluidly coupled to the gas recirculation loop; a vacuum protection device fluidly coupled to the gas recirculation loop; a volumetric flow device to measure a volume of the mixed-gas inhalant supplied to the patient; and a second connection to supply the mixed-gas inhalant to the patient. The system may also include: a first gas inlet to receive a regulated supply of the first gas having a first composition; a second gas inlet to receive a regulated supply of the second gas having a second composition; a first instrument fluidly coupled to the gas recirculation loop to measure one or more physical parameters of the mixed-gas inhalant; and a second instrument fluidly coupled to the gas recirculation loop to measure one or more compositional parameters of the mixed-gas inhalant.

According to example 23 there is provided a mixed-gas recirculation system. The system may include: a housing having an external surface that includes a transparent panel disposed in at least a portion of the external surface, the housing including an internal void space; a gas recirculation loop disposed at least partially within the housing, the gas recirculation loop including: a first connection to receive a first mixed-gas exhalant from a patient, the first mixed-gas exhalant including carbon dioxide at a first concentration, the first connection disposed on the external surface of the housing; a carbon dioxide removal device to remove at least a portion of the carbon dioxide included in the first mixed-gas exhalant to provide a second mixed-gas exhalant including carbon dioxide at a second concentration less than the first concentration, the carbon dioxide removal subsystem disposed at least partially within the housing; at least one gas inlet to receive at least one of a first gas and a second gas, the at least one gas inlet disposed on the external surface of the housing; a mixer to mix the second mixed-gas exhalant with a mixed-gas supply that includes the first gas and the second gas to provide a mixed-gas inhalant having a volumetric flow rate to the patient within a defined range, the mixer disposed at least partially within the housing; an overpressure protection device fluidly coupled to the gas recirculation loop and disposed at least partially within the housing; a vacuum protection device fluidly coupled to the gas recirculation loop and disposed at least partially within the housing; and a second connection to supply the mixed-gas inhalant to a patient, the patient supply connection disposed on the external surface of the housing.

According to example 38, there is provided a method of supplying a mixed-gas to a patient. The method may include: receiving, at a first connection to a gas recirculation loop, a first mixed-gas exhalant that includes carbon dioxide at a first concentration; removing, via a carbon dioxide removal device disposed in the gas recirculation loop, at least a portion of the carbon dioxide from the first mixed-gas exhalant to provide a second mixed-gas exhalant that includes carbon dioxide at a second concentration less than the first concentration; mixing the second mixed-gas exhalant with a mixed-gas supply that includes a first gas and a second gas to provide a mixed-gas inhalant at a volumetric flow rate within a defined range; measuring, via a volumetric flow device, the volumetric flow rate of the mixed-gas inhalant through the gas recirculation loop; measuring, via a first instrument, one or more physical parameters of the mixed-gas inhalant; measuring, via a second instrument, one or more compositional parameters of the mixed-gas inhalant; and supplying the inhalant to a second connection.

Example 2 may include elements of example 1 and the system may further include: at least one gas inlet connection to receive at least one of the first gas or the second gas.

Example 3 may include elements of any of examples 1 or 2 where the at least one gas inlet connection includes an oxygen inlet connection.

Example 4 may include elements of any of examples 1 through 3 where the at least one gas inlet connection further includes at least one of: a noble gas mixture inlet connection or an inert gas mixture inlet connection.

Example 5 may include elements of any of examples 1 through 4 where the noble gas mixture inlet connection comprises a heliox gas inlet connection.

Example 6 may include elements of any of examples 1 through 5 where the noble gas mixture inlet connection includes an oxygen/noble gas mixture inlet connection.

Example 7 may include elements of any of examples 1 through 6 where the oxygen/noble gas mixture inlet connection comprises an oxygen/heliox mixture inlet connection.

Example 8 may include elements of any of examples 1 through 7 where the overpressure protection device comprises a Positive End Expiratory Pressure (PEEP) valve.

Example 9 may include elements of any of examples 1 through 8 where the vacuum protection device comprises a Positive End Expiratory Pressure (PEEP) valve.

Example 10 may include elements of any of examples 1 through 9 where the second instrument comprises one or more instruments to measure the volumetric concentration of at least one of the first gas or the second gas in the mixed-gas inhalant.

Example 11 may include elements of any of examples 1 through 10 where the volumetric flow device comprises a respiration bag visible from one or more locations remote from the mixed-gas recirculation system.

Example 12 may include elements of any of examples 1 through 11 and the system may further include a scale to quantify the volume of the mixed-gas inhalant present in the respiration bag.

Example 13 may include elements of any of examples 1 through 12 where the first connection and the second connection comprise a single, dual limb circuit connector.

Example 14 may include elements of any of examples 1 through 13 where the gas recirculation loop further includes a heat and moisture exchanger (HME) fluidly coupled to the dual limb circuit connector.

Example 15 may include elements of any of examples 1 through 14 where the mixed-gas recirculation system comprises a modular system that includes: at least one first coupling to receive at least one of the first gas or the second gas; a second coupling to fluidly couple the gas recirculation loop to the first instrument; and a third coupling to fluidly couple the gas recirculation loop to the second instrument.

Example 16 may include elements of any of examples 1 through 15 where the gas recirculation loop further comprises a first biological HEPA filter fluidly coupled to the at least one first coupling.

Example 17 may include elements of any of examples 1 through 16 where the gas recirculation loop further comprises a second biological HEPA filter fluidly coupled inline proximate the volumetric flow device.

Example 18 may include elements of any of examples 1 through 17 where the gas recirculation loop further comprises a third biological HEPA filter fluidly coupled to the second coupling.

Example 19 may include elements of any of examples 1 through 18 where the gas recirculation loop further comprises a first backflow prevention device disposed inline with the second connection to prevent back flow of gas from the patient to the gas recirculation loop.

Example 20 may include elements of any of examples 1 through 19 where the gas recirculation loop further comprises a second backflow prevention device disposed inline with the first connection to prevent back flow of the first mixed-gas exhalant from the closed-loop gas recirculation system to the patient.

Example 21 may include elements of any of examples 1 through 20 and the system may further include control circuitry to: receive, from the first instrument, one or more input signals that include information indicative of the volumetric flow rate of the inhalant to the patient; and generate a control output signal to adjust the flow of at least one of the first gas or the second gas to the gas recirculation loop to maintain the volumetric flow rate of the mixed-gas inhalant to the patient within the defined range.

Example 22 may include elements of any of examples 1 through 21 where the gas recirculation loop further comprises a port fluidly coupled to the gas recirculation loop, the port to receive one or more therapeutic materials for introduction to the patient via the mixed-gas inhalant.

According to example 1, there is provided a mixed-gas recirculation system. The mixed-gas recirculation system may include: a gas recirculation loop that includes: a first connection to receive a first mixed-gas exhalant from a patient, the first mixed-gas exhalant having carbon dioxide at a first concentration; a carbon dioxide removal device to remove at least a portion of the carbon dioxide present in the first exhalant received from the patient to provide a second mixed-gas exhalant having carbon dioxide at a second volumetric concentration less than the first volumetric concentration; a mixer to combine the second exhalant with volumetrically controlled flowrate of a mixed-gas supply that includes a first gas and a second gas to provide a mixed-gas inhalant having a volumetric feed rate within a defined range; an overpressure protection device fluidly coupled to the gas recirculation loop; a vacuum protection device fluidly coupled to the gas recirculation loop; a volumetric flow device to measure a volume of the mixed-gas inhalant supplied to the patient; and a second connection to supply the mixed-gas inhalant to the patient. The system may also include: a first gas inlet to receive a regulated supply of the first gas having a first composition; a second gas inlet to receive a regulated supply of the second gas having a second composition; a first instrument fluidly coupled to the gas recirculation loop to measure one or more physical parameters of the mixed-gas inhalant; and a second instrument fluidly coupled to the gas recirculation loop to measure one or more compositional parameters of the mixed-gas inhalant.

According to example 23 there is provided a mixed-gas recirculation system. The system may include: a housing having an external surface that includes a transparent panel disposed in at least a portion of the external surface, the housing including an internal void space; a gas recirculation loop disposed at least partially within the housing, the gas recirculation loop including: a first connection to receive a first mixed-gas exhalant from a patient, the first mixed-gas exhalant including carbon dioxide at a first concentration, the first connection disposed on the external surface of the housing; a carbon dioxide removal device to remove at least a portion of the carbon dioxide included in the first mixed-gas exhalant to provide a second mixed-gas exhalant including carbon dioxide at a second concentration less than the first concentration, the carbon dioxide removal subsystem disposed at least partially within the housing; at least one gas inlet to receive at least one of a first gas and a second gas, the at least one gas inlet disposed on the external surface of the housing; a mixer to mix the second mixed-gas exhalant with a mixed-gas supply that includes the first gas and the second gas to provide a mixed-gas inhalant having a volumetric flow rate to the patient within a defined range, the mixer disposed at least partially within the housing; an overpressure protection device fluidly coupled to the gas recirculation loop and disposed at least partially within the housing; a vacuum protection device fluidly coupled to the gas recirculation loop and disposed at least partially within the housing; and a second connection to supply the mixed-gas inhalant to a patient, the patient supply connection disposed on the external surface of the housing.

According to example 38, there is provided a method of supplying a mixed-gas to a patient. The method may include: receiving, at a first connection to a gas recirculation loop, a first mixed-gas exhalant that includes carbon dioxide at a first concentration; removing, via a carbon dioxide removal device disposed in the gas recirculation loop, at least a portion of the carbon dioxide from the first mixed-gas exhalant to provide a second mixed-gas exhalant that includes carbon dioxide at a second concentration less than the first concentration; mixing the second mixed-gas exhalant with a mixed-gas supply that includes a first gas and a second gas to provide a mixed-gas inhalant at a volumetric flow rate within a defined range; measuring, via a volumetric flow device, the volumetric flow rate of the mixed-gas inhalant through the gas recirculation loop; measuring, via a first instrument, one or more physical parameters of the mixed-gas inhalant; measuring, via a second instrument, one or more compositional parameters of the mixed-gas inhalant; and supplying the inhalant to a second connection.

Example 24 may include elements of example 23 where the at least one gas inlet includes an inlet to receive a regulated oxygen supply.

Example 25 may include elements of any of examples 23 or 24 where the at least one gas inlet includes an inlet to receive a gas mixture containing at least one noble gas.

Example 26 may include elements of any of examples 23 through 25 where the at least one gas inlet includes an inlet to receive a heliox gas mixture.

Example 27 may include elements of any of examples 23 through 26 where the overpressure protection valve comprises a Positive End Expiratory Pressure (PEEP) valve.

Example 28 may include elements of any of examples 23 through 27 where the vacuum protection device comprises a Positive End Expiratory Pressure (PEEP) valve.

Example 29 may include elements of any of examples 23 through 28 where the transparent panel included in the external surface of the housing includes a scale to quantify the volume of air in the volumetric flow device.

Example 30 may include elements of any of examples 23 through 29 where the first connection and the second connection comprise a single, dual limb circuit connector.

Example 31 may include elements of any of examples 23 through 30 and the system may further include: a heat and moisture exchanger (HME) fluidly coupled to the dual limb circuit connector.

Example 32 may include elements of any of examples 23 through 31 and the system may further include: at least one first coupling to fluidly couple the gas recirculation loop to at least one of: a first gas supply or a second gas supply; a second coupling to fluidly couple the gas recirculation loop to an external first instrument to measure one or more physical parameters of the mixed-gas inhalant; and a third coupling to fluidly couple the gas recirculation loop an external second instrument to measure one or more compositional parameters of the mixed-gas inhalant.

Example 33 may include elements of any of examples 23 through 32 and the system may further include: a first biological HEPA filter fluidly coupled to the at least one first coupling.

Example 34 may include elements of any of examples 23 through 33 and the system may further include: a second biological HEPA filter fluidly coupled to the second coupling.

Example 35 may include elements of any of examples 23 through 34 and the system may further include: a third biological HEPA filter fluidly coupled to the gas recirculation loop disposed proximate the volumetric flow device.

Example 36 may include elements of any of examples 23 through 35 and the system may further include: a first backflow prevention device disposed proximate the first connection to prevent back flow of gas from the patient to the gas recirculation loop.

Example 37 may include elements of any of examples 23 through 36 and the system may further include: a second backflow protection device disposed inline with the patient return connection to prevent a reverse flow of first mixed-gas exhalant from the gas recirculation loop to the patient.

According to example 1, there is provided a mixed-gas recirculation system. The mixed-gas recirculation system may include: a gas recirculation loop that includes: a first connection to receive a first mixed-gas exhalant from a patient, the first mixed-gas exhalant having carbon dioxide at a first concentration; a carbon dioxide removal device to remove at least a portion of the carbon dioxide present in the first exhalant received from the patient to provide a second mixed-gas exhalant having carbon dioxide at a second volumetric concentration less than the first volumetric concentration; a mixer to combine the second exhalant with volumetrically controlled flowrate of a mixed-gas supply that includes a first gas and a second gas to provide a mixed-gas inhalant having a volumetric feed rate within a defined range; an overpressure protection device fluidly coupled to the gas recirculation loop; a vacuum protection device fluidly coupled to the gas recirculation loop; a volumetric flow device to measure a volume of the mixed-gas inhalant supplied to the patient; and a second connection to supply the mixed-gas inhalant to the patient. The system may also include: a first gas inlet to receive a regulated supply of the first gas having a first composition; a second gas inlet to receive a regulated supply of the second gas having a second composition; a first instrument fluidly coupled to the gas recirculation loop to measure one or more physical parameters of the mixed-gas inhalant; and a second instrument fluidly coupled to the gas recirculation loop to measure one or more compositional parameters of the mixed-gas inhalant.

According to example 23 there is provided a mixed-gas recirculation system. The system may include: a housing having an external surface that includes a transparent panel disposed in at least a portion of the external surface, the housing including an internal void space; a gas recirculation loop disposed at least partially within the housing, the gas recirculation loop including: a first connection to receive a first mixed-gas exhalant from a patient, the first mixed-gas exhalant including carbon dioxide at a first concentration, the first connection disposed on the external surface of the housing; a carbon dioxide removal device to remove at least a portion of the carbon dioxide included in the first mixed-gas exhalant to provide a second mixed-gas exhalant including carbon dioxide at a second concentration less than the first concentration, the carbon dioxide removal subsystem disposed at least partially within the housing; at least one gas inlet to receive at least one of a first gas and a second gas, the at least one gas inlet disposed on the external surface of the housing; a mixer to mix the second mixed-gas exhalant with a mixed-gas supply that includes the first gas and the second gas to provide a mixed-gas inhalant having a volumetric flow rate to the patient within a defined range, the mixer disposed at least partially within the housing; an overpressure protection device fluidly coupled to the gas recirculation loop and disposed at least partially within the housing; a vacuum protection device fluidly coupled to the gas recirculation loop and disposed at least partially within the housing; and a second connection to supply the mixed-gas inhalant to a patient, the patient supply connection disposed on the external surface of the housing.

According to example 38, there is provided a method of supplying a mixed-gas to a patient. The method may include: receiving, at a first connection to a gas recirculation loop, a first mixed-gas exhalant that includes carbon dioxide at a first concentration; removing, via a carbon dioxide removal device disposed in the gas recirculation loop, at least a portion of the carbon dioxide from the first mixed-gas exhalant to provide a second mixed-gas exhalant that includes carbon dioxide at a second concentration less than the first concentration; mixing the second mixed-gas exhalant with a mixed-gas supply that includes a first gas and a second gas to provide a mixed-gas inhalant at a volumetric flow rate within a defined range; measuring, via a volumetric flow device, the volumetric flow rate of the mixed-gas inhalant through the gas recirculation loop; measuring, via a first instrument, one or more physical parameters of the mixed-gas inhalant; measuring, via a second instrument, one or more compositional parameters of the mixed-gas inhalant; and supplying the inhalant to a second connection.

Example 39 may include elements of example 38 where mixing the second mixed-gas exhalant with a mixed-gas supply that includes a first gas and a second gas further comprises: mixing the second mixed-gas inhalant with a mixed-gas supply that includes a first gas containing oxygen and a second gas containing a noble gas.

Example 40 may include elements of any of examples 38 or 39 where mixing the second mixed-gas exhalant with the mixed-gas supply that includes the first gas containing oxygen and the second gas containing a noble gas further comprises: mixing the second mixed-gas exhalant with a mixed-gas supply that includes a first gas that includes oxygen and a second gas that includes a noble gas/oxygen mixture.

Example 41 may include elements of any of examples 38 through 40 and the method may further include releasing, in response to a pressure within the gas recirculation loop exceeding a defined maximum threshold value, the mixed-gas inhalant to atmosphere via one or more overpressure protection devices.

Example 42 may include elements of any of examples 38 through 41 and the method may further include: admitting, in response to a pressure within the gas recirculation loop falling below a defined minimum threshold value, atmospheric air into the gas recirculation loop via one or more vacuum protection devices.

Example 43 may include elements of any of examples 38 through 42 where measuring the volumetric flow of the mixed-gas inhalant through the gas recirculation loop further comprises: measuring the volumetric flow of the mixed-gas inhalant through the gas recirculation loop via a respiration bag.

Example 44 may include elements of any of examples 38 through 43 where receiving, at the first connection to the gas recirculation loop, a first mixed-gas exhalant and supplying a mixed-gas inhalant to a second connection further comprises: supplying the mixed-gas inhalant and receiving the first mixed-gas exhalant via a single, dual limb circuit connector.

Example 45 may include elements of any of examples 38 through 44 and the method may further include: passing the mixed-gas inhalant and the first mixed-gas exhalant through a heat and moisture exchanger (HME) fluidly coupled to the dual limb circuit connector.

Example 46 may include elements of any of examples 38 through 45 and the method may further include: filtering at least one of: the first gas or the second gas through a first biological HEPA filter prior to introducing the respective gas to the gas recirculation loop.

Example 47 may include elements of any of examples 38 through 46 and the method may include: filtering the mixed-gas inhalant through a second biological HEPA filter fluidly coupled to the gas recirculation loop proximate the volumetric flow device.

Example 48 may include elements of any of examples 38 through 47 and the method may further include: filtering the mixed-gas inhalant through a third biological HEPA filter fluidly coupled to the gas recirculation loop proximate the first instrument.

Example 49 may include elements of any of examples 38 through 48 and the method may further include: limiting reverse flow through the gas recirculation loop via a first backflow prevention device disposed proximate the second connection.

Example 50 may include elements of any of examples 38 through 49 and the method may further include: limiting reverse flow through the gas recirculation loop via a second backflow prevention device disposed proximate the first connection.

Example 51 may include elements of any of examples 38 through 50 and the method may further include: receiving, by control circuitry, one or more input signals from the second instrument, the one or more input signals including information indicative of the volumetric flow rate of the inhalant to the patient; and generating, by the control circuitry, a control output signal to adjust the flow of at least one of the first gas or the second gas to the gas recirculation loop to maintain the volumetric flow rate of the mixed-gas inhalant to the patient within the defined range.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed:

1. A mixed-gas recirculation system, comprising:
a gas recirculation loop that includes:
a first connection to receive a first mixed-gas exhalant from a patient, the first mixed-gas exhalant having carbon dioxide at a first concentration;
a carbon dioxide removal device to remove at least a portion of the carbon dioxide present in the first exhalant received from the patient to provide a second mixed-gas exhalant having carbon dioxide at a second volumetric concentration less than the first volumetric concentration;
a mixer to combine the second exhalant with volumetrically controlled flowrate of a mixed-gas supply that includes a first gas and a second gas to provide a mixed-gas inhalant having a volumetric feed rate within a defined range;
an overpressure protection device fluidly coupled to the gas recirculation loop;
a vacuum protection device fluidly coupled to the gas recirculation loop;
a volumetric flow device to measure a volume of the mixed-gas inhalant supplied to the patient; and
a second connection to supply the mixed-gas inhalant to the patient;
a first gas inlet to receive a regulated supply of the first gas having a first composition;
a second gas inlet to receive a regulated supply of the second gas having a second composition, the second composition different from the first composition;
a first instrument fluidly coupled to the gas recirculation loop to measure one or more physical parameters of the mixed-gas inhalant; and
a second instrument fluidly coupled to the gas recirculation loop to measure one or more compositional parameters of the mixed-gas inhalant.

2. The system of claim 1 further comprising at least one gas inlet connection to receive at least one of the first gas or the second gas.

3. The system of claim 2 wherein the at least one gas inlet connection includes an oxygen inlet connection.

4. The system of claim 3 wherein the at least one gas inlet connection further includes at least one of: a noble gas mixture inlet connection or an inert gas mixture inlet connection.

5. The system of claim 4 wherein the noble gas mixture inlet connection comprises a Heliox gas inlet connection.

6. The system of claim 2 wherein the noble gas mixture inlet connection includes an oxygen/noble gas mixture inlet connection.

7. The system of claim 6 wherein the oxygen/noble gas mixture inlet connection comprises an oxygen/Heliox mixture inlet connection.

8. The system of claim 1 wherein the overpressure protection device comprises a Positive End Expiratory Pressure (PEEP) valve.

9. The system of claim 1 wherein the vacuum protection device comprises a Positive End Expiratory Pressure (PEEP) valve.

10. The system of claim 1 wherein the second instrument comprises one or more instruments to measure the volumetric concentration of at least one of the first gas or the second gas in the mixed-gas inhalant.

11. The system of claim 1 wherein the volumetric flow device comprises a respiration bag visible from one or more locations remote from the mixed-gas recirculation system.

12. The system of claim 11 further comprising a scale to quantify the volume of the mixed-gas inhalant present in the respiration bag.

13. The system of claim 1 wherein the first connection and the second connection comprise a single, dual limb circuit connector.

14. The system of claim 13 wherein the gas recirculation loop further includes a heat and moisture exchanger (HME) fluidly coupled to the dual limb circuit connector.

15. The system of claim 1 wherein the mixed-gas recirculation system comprises a modular system that includes:
at least one first coupling to receive at least one of the first gas or the second gas;
a second coupling to fluidly couple the gas recirculation loop to the first instrument; and
a third coupling to fluidly couple the gas recirculation loop to the second instrument.

16. The system of claim 15 wherein the gas recirculation loop further comprises a first biological HEPA filter fluidly coupled to the at least one first coupling.

17. The system of claim 16 wherein the gas recirculation loop further comprises a second biological HEPA filter fluidly coupled inline proximate the volumetric flow device.

18. The system of claim 17 wherein the gas recirculation loop further comprises a third biological HEPA filter fluidly coupled to the second coupling

19. The system of claim 1 wherein the gas recirculation loop further comprises a first backflow prevention device disposed inline with the second connection to prevent back flow of gas from the patient to the gas recirculation loop.

20. The system of claim 19 wherein the gas recirculation loop further comprises a second backflow prevention device disposed inline with the first connection to prevent back flow of the first mixed-gas exhalant from the closed-loop gas recirculation system to the patient.

21. The system of claim 1 further comprising control circuitry to:
receive, from the first instrument, one or more input signals that include information indicative of the volumetric flow rate of the inhalant to the patient; and
generate a control output signal to adjust the flow of at least one of the first gas or the second gas to the gas recirculation loop to maintain the volumetric flow rate of the mixed-gas inhalant to the patient within the defined range.

22. The system of claim 1 wherein the gas recirculation loop further comprises a port fluidly coupled to the gas recirculation loop, the port to receive one or more therapeutic materials for introduction to the patient via the mixed-gas inhalant.

23. The system of claim 1, further comprising a spring loaded purge valve disposed between the first gas inlet and the mixer.

24. A mixed-gas recirculation system comprising:
a housing having an external surface that includes a transparent panel disposed in at least a portion of the external surface, the housing including an internal void space;
a gas recirculation loop disposed at least partially within the housing, the gas recirculation loop including:
a first connection to receive a first mixed-gas exhalant from a patient, the first mixed-gas exhalant including carbon dioxide at a first concentration, the first connection disposed on the external surface of the housing;
a carbon dioxide removal device to remove at least a portion of the carbon dioxide included in the first mixed-gas exhalant to provide a second mixed-gas exhalant including carbon dioxide at a second concentration less than the first concentration, the carbon dioxide removal subsystem disposed at least partially within the housing;
at least one gas inlet to receive at least one of a first gas and a second gas, the at least one gas inlet disposed on the external surface of the housing;
a mixer to mix the second mixed-gas exhalant with a mixed-gas supply that includes the first gas and the second gas to provide a mixed-gas inhalant having a volumetric flow rate to the patient within a defined range, the mixer disposed at least partially within the housing;
an overpressure protection device fluidly coupled to the gas recirculation loop and disposed at least partially within the housing;
a vacuum protection device fluidly coupled to the gas recirculation loop and disposed at least partially within the housing; and
a second connection to supply the mixed-gas inhalant to a patient, the patient supply connection disposed on the external surface of the housing.

25. The gas recirculation loop of claim 24 wherein the at least one gas inlet includes an inlet to receive a regulated oxygen supply.

26. The gas recirculation loop of claim 24 wherein the at least one gas inlet includes an inlet to receive a gas mixture containing at least one noble gas.

27. The gas recirculation loop of claim 26 wherein the at least one gas inlet includes an inlet to receive a Heliox gas mixture.

28. The gas recirculation loop of claim 24 wherein the overpressure protection valve comprises a Positive End Expiratory Pressure (PEEP) valve.

29. The gas recirculation loop of claim 24 wherein the vacuum protection device comprises a Positive End Expiratory Pressure (PEEP) valve.

30. The modular, closed-loop, gas recirculation system of claim 24 wherein the transparent panel included in the external surface of the housing includes a scale to quantify the volume of air in the volumetric flow device.

31. The gas recirculation loop of claim 24 wherein the first connection and the second connection comprise a single, dual limb circuit connector.

32. The gas recirculation loop of claim 31, further comprising a heat and moisture exchanger (HME) fluidly coupled to the dual limb circuit connector.

33. The mixed-gas recirculation system of claim 24 further comprising:
at least one first coupling to fluidly couple the gas recirculation loop to at least one of: a first gas supply or a second gas supply;
a second coupling to fluidly couple the gas recirculation loop to an external first instrument to measure one or more physical parameters of the mixed-gas inhalant; and
a third coupling to fluidly couple the gas recirculation loop an external second instrument to measure one or more compositional parameters of the mixed-gas inhalant.

34. The gas recirculation loop of claim 33 further comprising a first biological HEPA filter fluidly coupled to the at least one first coupling.

35. The gas recirculation loop of claim 34 further comprising a second biological HEPA filter fluidly coupled to the second coupling.

36. The gas recirculation loop of claim 35 further comprising a third biological HEPA filter fluidly coupled to the gas recirculation loop disposed proximate the volumetric flow device.

37. The gas recirculation loop of claim 24 further comprising a first backflow prevention device disposed proximate the first connection to prevent back flow of gas from the patient to the gas recirculation loop.

38. The gas recirculation loop of claim 24 further comprising a second backflow protection device disposed inline with the patient return connection to prevent a reverse flow of first mixed-gas exhalant from the gas recirculation loop to the patient.

39. A method of supplying a mixed-gas to a patient, the method comprising:
receiving, at a first connection to a gas recirculation loop, a first mixed-gas exhalant that includes carbon dioxide at a first concentration;
removing, via a carbon dioxide removal device disposed in the gas recirculation loop, at least a portion of the carbon dioxide from the first mixed-gas exhalant to provide a second mixed-gas exhalant that includes carbon dioxide at a second concentration less than the first concentration;
mixing the second mixed-gas exhalant with a mixed-gas supply that includes a first gas and a second gas to provide a mixed-gas inhalant at a volumetric flow rate within a defined range;
measuring, via a volumetric flow device, the volumetric flow rate of the mixed-gas inhalant through the gas recirculation loop;
measuring, via a first instrument, one or more physical parameters of the mixed-gas inhalant;
measuring, via a second instrument, one or more compositional parameters of the mixed-gas inhalant; and
supplying the inhalant to a second connection.

40. The method of claim 39 wherein mixing the second mixed-gas exhalant with a mixed-gas supply that includes a first gas and a second gas further comprises:
mixing the second mixed-gas inhalant with a mixed-gas supply that includes a first gas containing oxygen and a second gas containing a noble gas.

41. The method of claim 40 wherein mixing the second mixed-gas exhalant with the mixed-gas supply that includes the first gas containing oxygen and the second gas containing a noble gas further comprises:
mixing the second mixed-gas exhalant with a mixed-gas supply that includes a first gas that includes oxygen and a second gas that includes a noble gas/oxygen mixture.

42. The method of claim 39 further comprising:
releasing, in response to a pressure within the gas recirculation loop exceeding a defined maximum threshold value, the mixed-gas inhalant to atmosphere via one or more overpressure protection devices.

43. The method of claim 42 further comprising:
admitting, in response to a pressure within the gas recirculation loop falling below a defined minimum threshold value, atmospheric air into the gas recirculation loop via one or more vacuum protection devices.

44. The method of claim 39 wherein measuring the volumetric flow of the mixed-gas inhalant through the gas recirculation loop further comprises:
measuring the volumetric flow of the mixed-gas inhalant through the gas recirculation loop via a respiration bag.

45. The method of claim 39 wherein receiving, at the first connection to the gas recirculation loop, a first mixed-gas exhalant and supplying a mixed-gas inhalant to a second connection further comprises:
supplying the mixed-gas inhalant and receiving the first mixed-gas exhalant via a single, dual limb circuit connector.

46. The method of claim 45, further comprising:
passing the mixed-gas inhalant and the first mixed-gas exhalant through a heat and moisture exchanger (HME) fluidly coupled to the dual limb circuit connector.

47. The method of claim 39, further comprising:
filtering at least one of: the first gas or the second gas through a first biological HEPA filter prior to introducing the respective gas to the gas recirculation loop.

48. The method of claim 47, further comprising:
filtering the mixed-gas inhalant through a second biological HEPA filter fluidly coupled to the gas recirculation loop proximate the volumetric flow device.

49. The method of claim 48, further comprising:
filtering the mixed-gas inhalant through a third biological HEPA filter fluidly coupled to the gas recirculation loop proximate the first instrument.

50. The method of claim 39, further comprising:
limiting reverse flow through the gas recirculation loop via a first backflow prevention device disposed proximate the second connection.

51. The method of claim 50, further comprising:
limiting reverse flow through the gas recirculation loop via a second backflow prevention device disposed proximate the first connection.

52. The method of claim 39, further comprising:
receiving, by control circuitry, one or more input signals from the second instrument, the one or more input signals including information indicative of the volumetric flow rate of the inhalant to the patient; and
generating, by the control circuitry, a control output signal to adjust the flow of at least one of the first gas or the second gas to the gas recirculation loop to maintain the volumetric flow rate of the mixed-gas inhalant to the patient within the defined range.
